(19) United States
(12) Patent Application Publication
(10) Pub. No.: US 2022/0039815 A1
(43) Pub. Date: Feb. 10, 2022

(54) AUTOMATICALLY-LOCKING VACUUM SYRINGES, AND ASSOCIATED SYSTEMS AND METHODS

(71) Applicant: Inari Medical, Inc., Irvine, CA (US)

(72) Inventors: John Coleman Thress, Capistrano Beach, CA (US); Evan Stuart Hunter, Mission Viejo, CA (US); Christopher Andrew Zikry, Northridge, CA (US); Kendall Anne Ruggles, Newport Beach, CA (US); Benjamin Edward Merritt, San Clemente, CA (US); Brian Michael Strauss, San Clemente, CA (US)

(21) Appl. No.: 17/396,426

(22) Filed: Aug. 6, 2021

Related U.S. Application Data
(60) Provisional application No. 63/061,902, filed on Aug. 6, 2020.

(51) Int. Cl. A61B 17/22; A61M 1/00

(52) U.S. Cl. CPC A61B 17/22; A61M 1/67; A61M 2205/584; A61B 2017/22079; A61M 1/84

(57) ABSTRACT

Automatically-locking syringes are disclosed herein. A syringe in accordance with embodiments of the present technology can include (i) a barrel, (ii) a plunger slidably positioned within the barrel, and (iii) a lock plate coupled to the barrel. The plunger also extends through an opening in the lock plate, and a biasing member is configured to bias the lock plate to a locking position. When the plunger is moved from a depressed position to a withdrawn position, the lock feature engages the lock plate to drive the lock plate away from the locking position to thereby permit the lock feature to pass through the opening in the lock plate. After the lock feature passes through the opening, the biasing member drives the lock plate to the locking position to inhibit movement of the plunger from the withdrawn position to the depressed position.

### CROSS-REFERENCE TO RELATED APPLICATION

This application claims the benefit of U.S. Provisional Patent Application No. 63/061,902, filed Aug. 6, 2020, and titled “AUTOMATICALLY-LOCKING VACUUM SYRINGES, AND ASSOCIATED SYSTEMS AND METHODS,” which is incorporated herein by reference in its entirety.

### TECHNICAL FIELD

The present technology relates generally to systems, methods, and devices for the intravascular treatment of emboli and/or thrombi within a blood vessel of a human patient. In particular, some embodiments of the present technology relate to automatically-locking syringes for generating and releasing stored vacuum pressure to aspirate clot material from a blood vessel.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long distance air travel, immobility) and clotting (e.g., cancer, recent surgery such as orthopedic surgery). DVT can obstruct drainage of venous blood from the legs leading to swelling, ulcers, pain and infection. DVT can also create a reservoir in which blood clots can collect and then travel to other parts of the body including the heart, lungs, brain (stroke), abdominal organs, and/or extremities.

In the pulmonary circulation, the undesirable material can cause harm by obstructing pulmonary arteries—a condition known as pulmonary embolism. If the obstruction is upstream, in the main or large branch pulmonary arteries, it can severely compromise total blood flow within the lungs, and therefore the entire body. This can result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. Embolectomies, for example, are a surgical technique involving incising a blood vessel and placing a balloon-tipped device (such as the Fogarty catheter) at the location of the occlusion. The balloon is then inflated at a point beyond the clot and used to withdraw the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and restenosis regularly occurs after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhaging, and in many patients the thrombolytic agents cannot be used at all.

Various devices exist for performing a thrombectomy or removing other foreign material. However, such devices have been found to have structures which are either highly complex, cause trauma to the treatment vessel, or lack the ability to be appropriately fixed against the vessel. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all the clot material.

Thus, there exists a need for improved systems and methods for embolic extraction.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIGS. 1A and 1B are an exploded isometric view and a side view, respectively, of a vacuum-pressure locking syringe in accordance with embodiments of the present technology.

FIGS. 2A and 2B are isometric views of the syringe of FIGS. 1A and 1B in a depressed position and a withdrawn position, respectively, in accordance with embodiments of the present technology.

FIG. 3A is a side view, and FIGS. 3B and 3C are enlarged side views, of the syringe illustrating the steps of locking a plunger of the syringe to a lock member of the syringe in accordance with embodiments of the present technology.

FIG. 4 is an enlarged isometric view of a portion of the syringe in accordance with additional embodiments of the present technology.

FIGS. 5A and 5B are an isometric view and a side view, respectively, of a syringe in a withdrawn position in accordance with additional embodiments of the present technology.

FIG. 5C is an isometric of the syringe of FIGS. 5A and 5B in a depressed position in accordance with embodiments of the present technology.

FIG. 6A is a side view of a vacuum-pressure locking syringe in accordance with additional embodiments of the present technology.

FIGS. 6B and 6C are enlarged side views of a locking mechanism of the syringe of FIG. 6A in a first position and a second position, respectively, in accordance with embodiments of the present technology.

FIG. 7 is a partially schematic side view of a clot treatment or clot removal system incorporating the syringe of FIGS. 1A-4, the syringe of FIGS. 5A-5C, and/or the syringe of FIGS. 6A-6C in accordance with embodiments of the present technology.

FIGS. 8A and 8B are enlarged, partially-schematic side views of a vacuum indicator of the clot removal system of FIG. 7 in a vacuum-off position and a vacuum-on position, respectively, in accordance with embodiments of the present technology.

FIG. 9 is a perspective view of a vacuum indicator in a vacuum-off position in accordance with additional embodiments of the present technology.

FIGS. 10A and 10B are perspective views of a vacuum indicator in a vacuum-off position and a vacuum-on position, respectively, in accordance with additional embodiments of the present technology.

FIG. 11 is a side-view of the syringe of FIGS. 1A and 1B including a vacuum indicator in accordance with embodiments of the present technology.

FIGS. 12A and 12B are perspective views of a syringe including a vacuum indicator in accordance with additional embodiments of the present technology.

FIGS. 13A-13C are side views of a vacuum indicator in a vacuum-off position, a partial-vacuum position, and a full-vacuum position, respectively, in accordance with additional embodiments of the present technology.

FIGS. 14A and 14B are an isometric view and a top view of a syringe in accordance with additional embodiments of the present technology.

FIGS. 14C and 14D are enlarged side views of a vacuum indicator of the syringe of FIGS. 14A and 14B in a “vacuum-off” and a “vacuum-on” position, respectively, in accordance with embodiments of the present technology.

FIGS. 14E and 14F are enlarged partially transparent side views of the vacuum indicator of FIGS. 14A-14D in the vacuum-off and the vacuum-on position, respectively, in accordance with embodiments of the present technology.

FIG. 14G is an enlarged side cross-sectional view of the vacuum indicator of FIGS. 14A-14F in the vacuum-off position in accordance with embodiments of the present technology.

FIGS. 15A and 15B are an isometric view and a side view of a syringe in accordance with additional embodiments of the present technology.

FIGS. 15C and 15D are enlarged side views of a vacuum indicator of the syringe of FIGS. 15A and 15B in a “vacuum-off” and a “vacuum-on” position, respectively, in accordance with embodiments of the present technology.

FIGS. 15E and 15F are enlarged partially transparent side views of the vacuum indicator of FIGS. 15A-15D in the vacuum-off and the vacuum-on position, respectively, in accordance with embodiments of the present technology.

FIG. 15G is an enlarged side cross-sectional view of the vacuum indicator of FIGS. 15A-15F in the vacuum-off position in accordance with embodiments of the present technology.

FIG. 16 is an enlarged side cross-sectional view of the vacuum indicator of FIGS. 15A-15G further including a biasing member in accordance with embodiments of the present technology.

### DETAILED DESCRIPTION

The present technology is generally directed to automatically-locking syringes, such as for use in clot removal systems for aspirating clot material from a blood vessel of a human patient. In some embodiments, an automatically-locking syringe can include (i) a barrel, (ii) a lock plate coupled to the barrel, and (iii) a plunger slidably positioned within the barrel and having a lock feature configured to engage the lock plate. The plunger also extends through an opening in the lock plate, and a biasing member is configured to bias the lock plate toward a locking position. When the plunger is moved through the barrel from a depressed position to a withdrawn position, the lock feature is configured to engage the lock plate to drive the lock plate away from the locking position to thereby permit the lock feature to pass through the opening in the lock plate. When the lock feature passes through the opening, the biasing member is configured to bias the lock plate to the locking position to inhibit movement of the plunger from the withdrawn position to the depressed position. Accordingly, in one aspect of the present technology, the plunger is automatically locked in position via engagement of the lock plate with the lock feature when the plunger is withdrawn a selected distance.

In additional embodiments, an automatically-locking syringe can include (i) a barrel having a flange, (ii) a plunger slidably positioned within the barrel, and (iii) at least one lock member coupled to the plunger. The lock member can include a body and a first arm hingedly coupled to the body. The first arm is configured to be biased at least partially outwardly away from a longitudinal axis of the plunger to a locking position. When the plunger is in a withdrawn position, the first arm can engage the flange of the barrel to inhibit movement of the plunger through the barrel from the withdrawn position to a depressed position. The syringe can further include an actuator that is movable between a first position and a second position. The actuator can include a second arm configured to engage the first arm in the second position to drive the first arm inwardly toward the longitudinal axis and away from the locking position. Accordingly, when the plunger is withdrawn, moving the actuator from the first position to the second position can drive the first arm radially inward away from the locking position to permit movement of the plunger through the barrel from the withdrawn position to the depressed position.

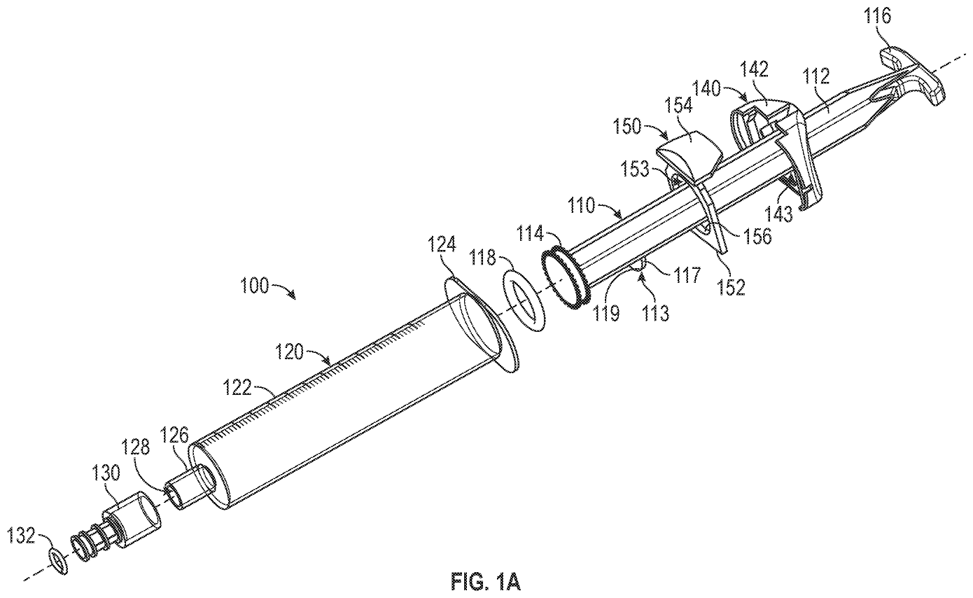

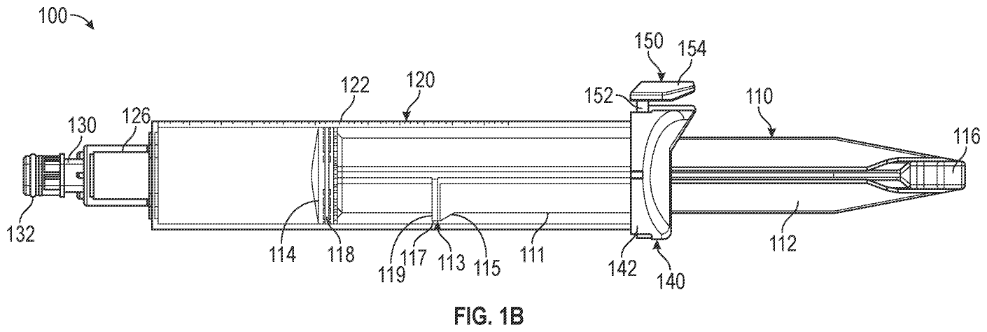

Specific details of several embodiments of the present technology are described herein with reference to FIGS. 1A-16. The present technology, however, may be practiced without some of these specific details. In some instances, well-known structures and techniques often associated with the disclosed syringes, clot removal systems, and the like have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements may be arbitrarily enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. SELECTED EMBODIMENTS OF VACUUM-PRESSURE LOCKING SYRINGES

FIGS. 1A and 1B are an exploded isometric view and a side view, respectively, of a vacuum-pressure locking syringe (“syringe 100”) in accordance with embodiments of the present technology. The syringe 100 is in a partially-withdrawn position in FIG. 1B. Referring to FIGS. 1A and 1B together, the syringe 100 includes a plunger 110 slidably positioned within a barrel 120. The barrel 120 is shown as partially transparent in FIG. 1B for clarity. The barrel 120 can include a barrel portion 122 (e.g., a cylindrical portion) extending between a flange 124 and a tip 126. In some embodiments, the barrel portion 122 can have a volume of about 60 cc or greater than 60 cc. The tip 126 is configured to be releasably or permanently coupled to an adaptor 130 (shown as partially transparent in FIGS. 1A and 1B). In some embodiments, the tip 126 can define a bore 128 having a size of about 26 French or greater. In the illustrated embodiment, the adaptor 130 is a Toomey-tip adaptor having a sealing member 132 (e.g., an O-ring) extending around an exterior surface thereof for sealingly engaging (e.g., connecting to) a Toomey fitting or Toomey adaptor. In other embodiments, the adaptor 130 can be omitted and the tip 126 of the barrel 120 can be directly coupled to another device or system (not shown), and/or the tip 126 can be another type of tip such as, for example, a Luer lock, Lock slip, and/or needle.

In the illustrated embodiment, the plunger 110 includes a shaft 112 extending between a flange 114 and a grip portion 116. The grip portion 116 is configured to be grasped by a user for withdrawing (e.g., retracting, pulling) and/or depressing (e.g., advancing, pushing) the plunger 110 through the barrel 120 during operation of the syringe 100. The plunger 110 can include a sealing member 118 positioned around the flange 114 and configured to sealingly engage an interior surface of the barrel portion 122 to define a sealed volume (e.g., of negative/vacuum pressure) within the barrel 120. The various components of the syringe 100 can comprise metal, plastic, and/or other suitable materials.

As best seen in FIG. 1B, the shaft 112 of the plunger 110 includes a lower edge or surface 111 and a lock feature 113 extending/protruding from the lower surface 111. In the illustrated embodiment, the lock feature 113 includes (i) a ramp portion 115, (ii) a plateau portion 117 extending from the ramp portion 115, and (iii) a stop surface 119 extending from the plateau portion 117. The ramp portion 115 can extend/slope at angle relative to the lower surface 111 in a direction (i) away from the lower surface 111 and (ii) toward the flange 114 of the plunger 110. The plateau portion 117 can extend away from the ramp portion 115 in a direction toward the flange 114 and generally parallel to the lower surface 111. The stop surface 119 can extend away from the plateau portion 117 in a direction generally perpendicular to the plateau portion 117 and the lower surface 111. In some embodiments, the lock feature 113 can be a tab, flat face, half disc, and/or other feature extending from the lower surface 111 of the shaft 112.

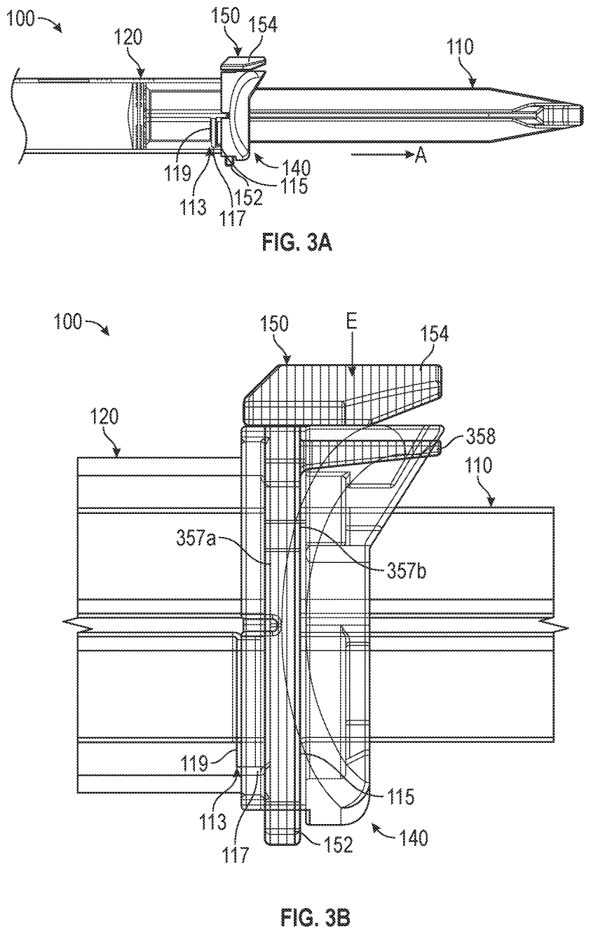

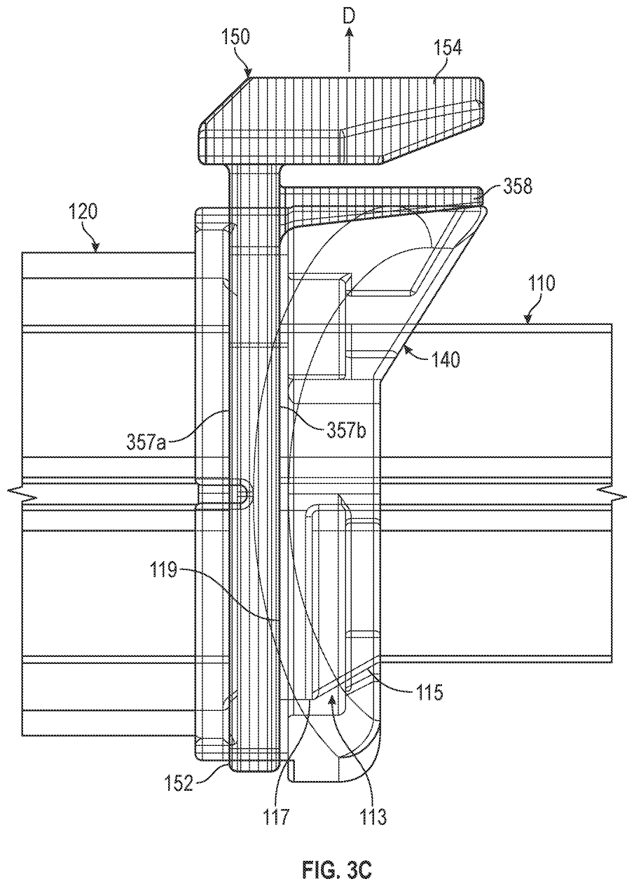

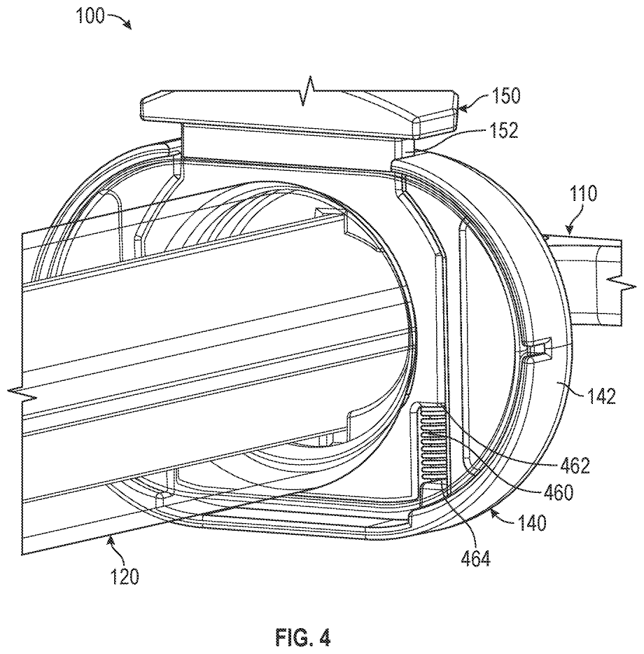

In the illustrated embodiment, the syringe 100 further includes a base 140 and a lock member 150 operably coupled to the base 140. In some embodiments, the base 140 can be coupled to the flange 124 of the barrel 120. Accordingly, the base 140 can couple the lock member 150 to the barrel 120. The base 140 can include a body 142 having an opening 143 extending therethrough and configured to slidably receive the shaft 112 of the plunger 110. The lock member 150 can include a lock plate 152 coupled to a button portion 154. The lock plate 152 can include an opening 153 (i) extending therethrough, (ii) defined by an edge portion 156, and (iii) configured to slidably receive the shaft 112 of the plunger 110. In some embodiments, the lock member 150 can be movably coupled to the base 140 via, for example, a biasing member (e.g., an arm 358 illustrated in FIGS. 3B and 3C, springs 460 illustrated in FIG. 4, and/or another type of biasing member). As described in greater detail below with reference to FIGS. 3A-4, the lock plate 152 is configured to engage the lock feature 113 of the plunger 110 when the plunger 110 is withdrawn to a selected position to lock the plunger 110 in the selected position. The selected position can correspond to a pre-selected vacuum volume of the barrel 120. The button portion 154 is actuatable (e.g., depressible) by a user to release the lock plate 152 from the lock feature 113 to release the plunger 110 to, for example, permit the plunger 110 to move through the barrel 120 to expel any contents (e.g., bodily fluids such as blood and clot material) collected in the barrel 120 from the tip 126.

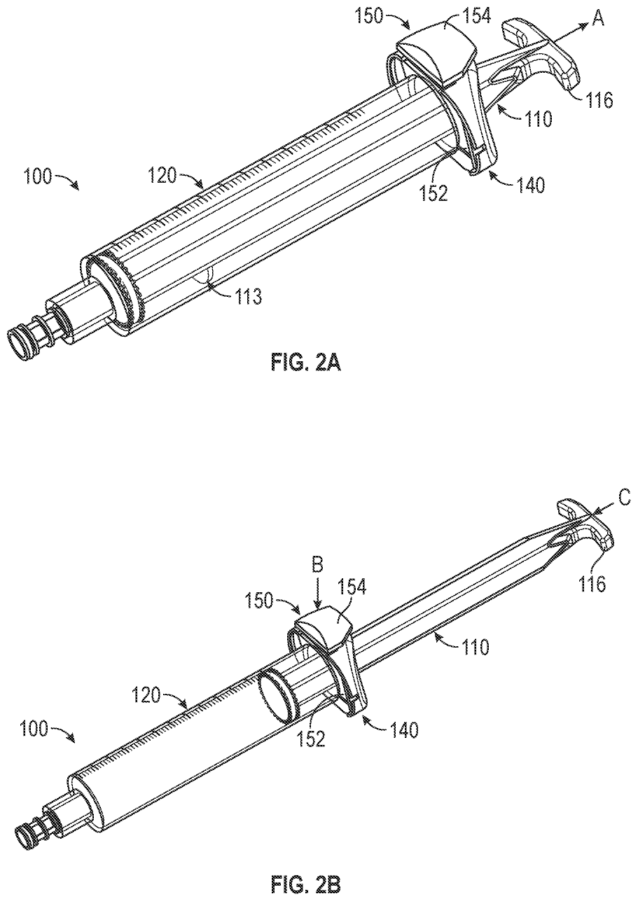

FIGS. 2A and 2B are isometric views of the syringe 100 in a depressed position (e.g., a first position, an emptied positioned, zero-vacuum position) and a withdrawn position (e.g., a second position, a vacuum-storage position, a vacuum-generation position, full-vacuum position), respectively, in accordance with embodiments of the present technology. The barrel 120 is shown as partially transparent in FIGS. 2A and 2B for clarity. Referring to FIGS. 2A and 2B together, in general, a user can move the syringe 100 from the depressed position to the withdrawn position by pulling the grip portion 116 of the plunger 110 in the direction of arrow A (FIG. 2A) relative to the barrel 120 to withdraw the plunger 110 through the barrel 120, the base 140, and the lock member 150. In some embodiments, withdrawing the plunger 110 can charge a vacuum (e.g., generate negative pressure) within the barrel 120. Moreover, the lock plate 152 of the lock member 150 can engage the lock feature 113 (obscured in FIG. 2B) of the plunger 110 when the plunger 110 is withdrawn past the lock plate 152 to automatically lock the plunger 110 in the withdrawn position. To move the syringe 100 from the withdrawn position to the depressed position, the user can (i) actuate the button portion 154 of the lock member 150 (e.g., depress the button portion 154 in the direction of arrow B in FIG. 2B) to disengage the lock plate 152 from the lock feature 113 to unlock the plunger 110 from the lock member 150 and then (ii) push the plunger 110 in the direction of arrow C (FIG. 2B) relative to the barrel 120.

FIG. 3A is a side view, and FIGS. 3B and 3C are enlarged side views, of the syringe 100 illustrating the process of locking the plunger 110 to the lock member 150 in accordance with embodiments of the present technology. The syringe 100 is in a partially-withdrawn position in FIGS. 3A and 3B. More specifically, FIGS. 3A and 3B illustrate the syringe 100 as the lock feature 113 passes through the opening 153 (FIG. 1A) in the lock member 150 during withdrawal of the plunger 110 from the depressed position to the withdrawn position. FIG. 3C illustrates the syringe 100 after the lock feature 113 has been withdrawn through the opening 153 past the lock plate 152. The barrel 120 is shown as partially transparent in FIGS. 3A-3C, and the base 140 is shown as partially transparent in FIGS. 3B and 3C for clarity.

Referring to first to FIGS. 3B and 3C together, the lock member 150 further includes an arm 358 (e.g., a biasing member) operably coupling the lock member 150 to the base 140. In the illustrated embodiment, for example, the arm 358 projects away from (e.g., perpendicular) to the lock plate 152 and presses against (e.g., engages, attaches to) the base 140. The arm 358 can be a spring arm, living hinge, lever arm, or other member that is (i) hingedly/movably coupled to the lock member 150 and (ii) configured to bias the lock plate 152 upward to a locking position (e.g., a first position) as shown in FIGS. 3C and 1B (e.g., in an upward direction indicated by arrow D in FIG. 3C). As the lock feature 113 is withdrawn past the lock plate 152, the lock feature 113 can engage the lock plate 152 and drive the lock plate 152 against the biasing force of the arm 358 away from the locking position (e.g., toward a second position) as shown in FIG. 3B (e.g., in a downward direction indicated by arrow E in FIG. 3B).

More specifically, referring to FIGS. 3A and 3B together, as the plunger 110 is withdrawn toward the lock member 150 in the direction of the arrow A, the ramp portion 115 of the lock feature 113 first engages the edge portion 156 (FIG. 1A) of the lock plate 152 at a first side 357a of the lock plate 152 opposite a second side 357b. As the plunger is withdrawn farther, the angled ramp portion 115 drives the lock plate 152 (and the button portion 154) downward in the direction of the arrow E against the biasing force of the arm 358 (e.g., away from the locking position) until the lock plate 152 reaches the plateau portion 117 of the lock feature 113. That is, the ramp portion 115 of the lock feature 113 translates the longitudinal force of the plunger 110 into radial movement of the lock plate 152. Continued withdrawal of the plunger 110 slides the lock plate 152 over/against the plateau portion 117 until the lock feature 113 reaches the stop surface 119. Referring to FIG. 3C, when the lock plate 152 passes the end of the plateau portion 117, the arm 358 can drive the lock plate 152 upward in the direction of arrow D to the locking position. The plunger 110 is then inhibited from moving/advancing past the lock plate 152 toward the depressed position (e.g., in the direction of the arrow C shown in FIG. 2B). Specifically, the second side 357b of the lock plate 152 can engage/contact the stop surface 119 of the lock feature 113 to inhibit advancement of the plunger 110. In other embodiments, the lock feature 113 and/or the lock plate 152 can have different configurations/arrangements that facilitate locking of the plunger 110 in the withdrawn position. For example, in some embodiments the plateau portion 117 of the lock feature 113 can be omitted.

To unlock the plunger 110 from the lock member 150, the user can actuate the button portion 154 of the lock member 150 by, for example, pressing the button portion 154 downward in the direction of arrow E. The movement of the button portion 154 drives the lock plate 152 downward relative to the lock feature 113 until the edge portion 156 (FIG. 1A) of the lock plate 152 fully passes the stop surface 119 of lock feature 113, thereby permitting movement of the lock feature 113 through the opening 153 (FIG. 1A) of the lock plate 152. The user can then advance the plunger 110 through the barrel 120 to expel any contents collected in the barrel 120.

Accordingly, in one aspect of the present technology, the plunger 110 is automatically locked in position—via the lock plate 152 and lock feature 113—when the plunger 110 is withdrawn a selected distance. In contrast, many conventional locking syringes require the user to rotate the plunger relative to the barrel to facilitate locking of the plunger. Such rotation can be difficult when the syringe has a large volume—for example, 60 cc or greater—and thus requires a relatively significant retraction force to withdraw the plunger. Therefore, the syringe 100 of the present technology has improved usability compared to conventional locking syringes, especially during procedures that require multiple withdrawals of the plunger 110 over the course of the procedure. In some embodiments, the lock member 150 and the lock feature 113 can be configured (e.g., shaped, positioned) such that the syringe 100 automatically locks at a selected position corresponding to a selected volume of the barrel 120. In some embodiments, the selected volume can be about 60 cc or greater.

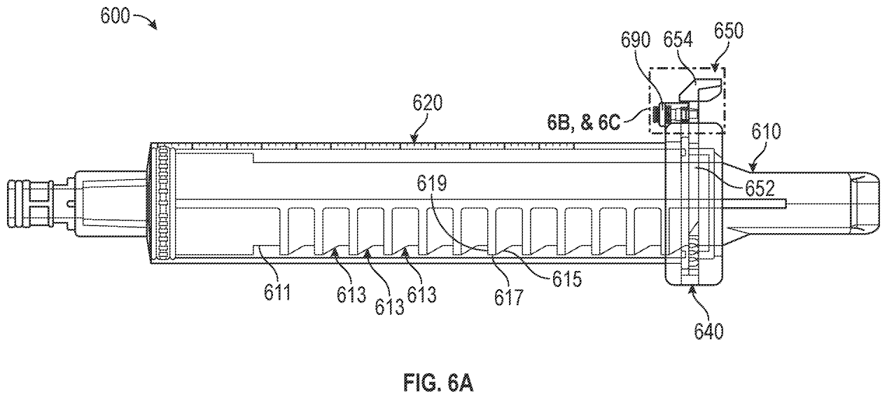

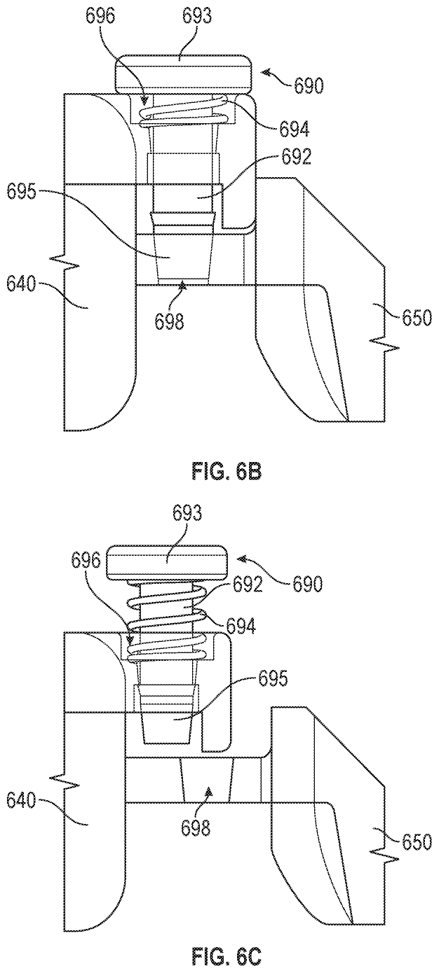

In another aspect of the present technology, the syringe 100 is lockable at only a single position. This can be particularly useful where the syringe 100 is used in procedures in which a single operational volume is preselected/desired, such as during a clot removal procedure that includes aspirating clot material from a vessel, as described in greater detail below with reference to FIG. 6.

FIG. 4 is an enlarged isometric view of a portion of the syringe 100 in accordance with additional embodiments of the present technology. The barrel 120 is shown as partially transparent in FIG. 4 for clarity. In the illustrated embodiment, the lock plate 152 of the lock member 150 is operably coupled to the base 140 via a pair of springs 460 (only one of the springs 460 is visible in FIG. 4). The springs 460 are configured to bias the lock plate 152 to the first position shown in FIGS. 4 and 1B, and can replace or supplement the arm 358 described in detail above with reference to FIGS. 3A-3C. More particularly, each of the springs 460 can be a compression spring operably coupled between a first spring mount 462 of the lock plate 152 and a second spring mount 464 of the body 142 of the base 140. In a similar manner as described in detail above with reference to FIGS. 3A-3C, (i) the lock feature 113 can engage the lock plate 152 and drive the lock plate 152 against the biasing force of the springs 460 during withdrawal of the plunger 110, and (ii) the springs 460 can drive the lock plate 152 upward after the lock feature 113 passes through the lock plate 152 to automatically lock the plunger 110 in the withdrawn position.

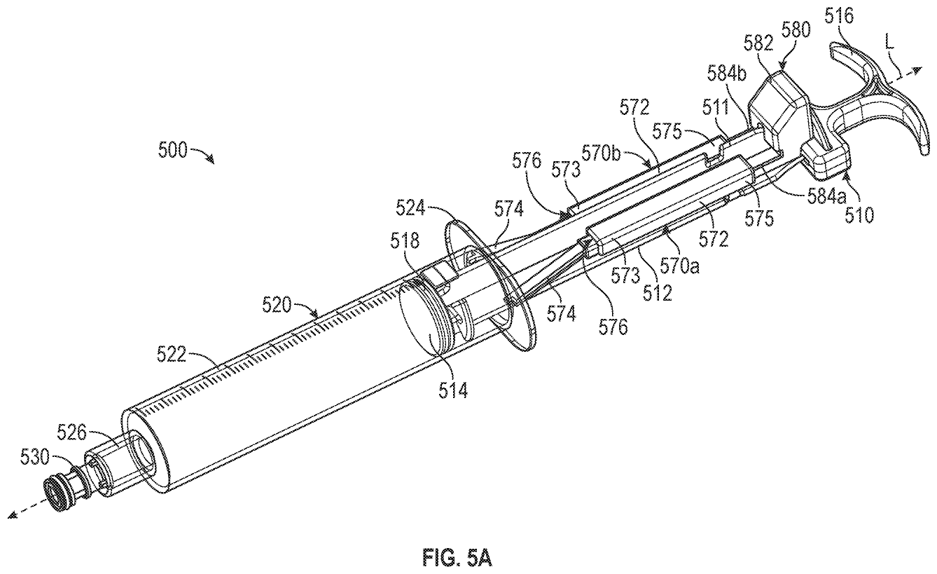

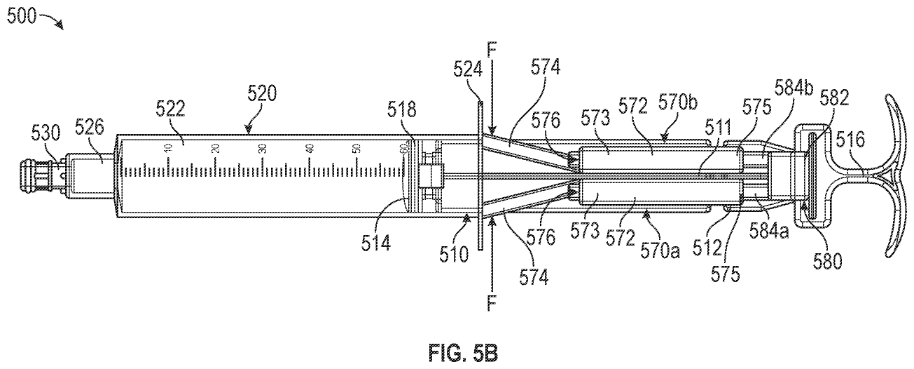

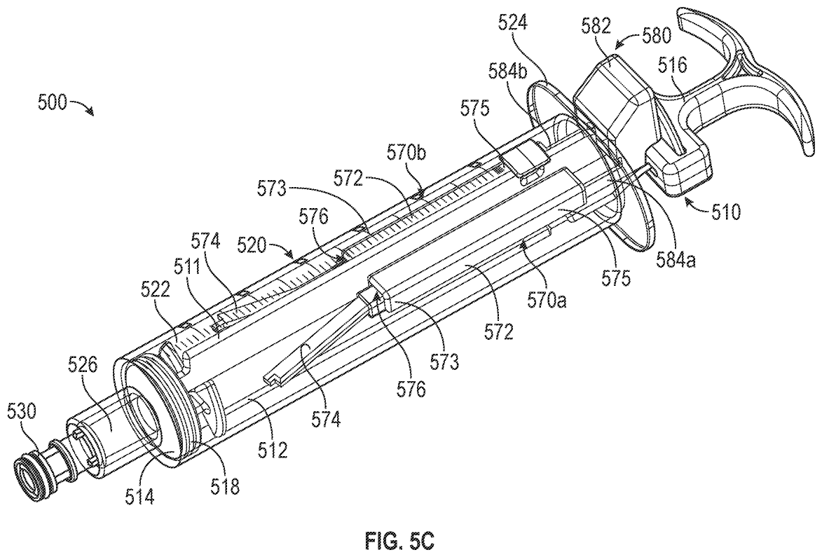

FIGS. 5A-5C are an isometric view, a side view, and another isometric view, respectively, of a syringe 500 in accordance with additional embodiments of the present technology. The syringe 500 is in a withdrawn/retracted position in FIGS. 5A and 5B, and in a depressed/advanced position in FIG. 5C. Referring to FIGS. 5A-5C together, the syringe 500 can include several features generally similar or identical to the syringe 100 described in detail above with reference to FIGS. 1A-4. In the illustrated embodiment, for example, the syringe 500 includes a plunger 510 configured to be slidably positioned within a barrel 520. The barrel 520 is shown as partially transparent in FIGS. 5A-5C for clarity. The barrel 520 can include a barrel portion 522 extending between a flange 524 and a tip 526. In some embodiments, the tip 526 is configured to be releasably or permanently coupled to an adaptor 530 (e.g., a Toomey tip adaptor; shown as partially transparent in FIGS. 5A-5C). The plunger 510 can include a shaft 512 extending between a flange 514 and a grip portion 516. The plunger 510 can include a sealing member 518 positioned around the flange 514 and configured to sealingly engage an interior surface of the barrel portion 522 to define a sealed volume (e.g., of negative/vacuum pressure) within the barrel 520.

In the illustrated embodiment, the syringe 500 further includes a pair of lock members 570 (identified individually as a first lock member 570a and a second lock member 570b) operably coupled to an actuator 580. In some embodiments, the shaft 512 of the plunger 510 includes a spline 511 (e.g., a wall portion), and the lock members 570 can be positioned on opposite sides of the spline 511. The lock members 570 can each include a body 572 and an arm 574 hingedly/movably attached to the body 572. The arms 574 can be spring arms, living hinges, lever arms, or other members configured to be biased outwardly away from a longitudinal axis L (FIG. 5A) of the syringe 500. The bodies 572 can each include a first end portion 573 and a second end portion 575 opposite the first end portion 573, and can each include/define an elongate opening 576 extending between the first and second end portions 573, 575. The lock members 570 can be formed of plastic, metal, and/or other suitable materials and can be single integral parts (e.g., injected molded parts) or can be formed of discrete parts coupled together.

In the illustrated embodiment, the actuator 580 includes a push portion 582 and a pair of elongate arms 584 (identified individually as a first arm 584a and a second arm 584b) extending away from the push portion 582. In some embodiments, the push portion 582 is slidably coupled to the spline 511 of the shaft 512 and configured to slide at least partially between (i) a first position in which the push portion 582 abuts and/or is adjacent to the grip portion 516 of the plunger 510 and (ii) a second position in which the push portion 582 abuts and/or is adjacent to the second end portions 575 of the arms 574. The plunger 580 is shown in the second position in FIGS. 5A-5C. The first arm 584a can extend at least partially through the opening 576 in the first body 570a, and the second arm 584b can extend at least partially through the opening 576 in the second body 570b. In some embodiments, the arms 584 are slidably positioned within the openings 576 such that movement of the push portion 582 between the first and second positions advances/retracts the arms 584 through the openings 576. In some embodiments, in the first position, the arms 584 of the actuator 580 do not extend over the arms 574 of the lock members 570. In the second position, the arms 584 of the actuator 580 can extend over/around all or a portion of the arms 574 of the lock members 570 to move (e.g., pinch) the arms 574 inwardly toward the longitudinal axis L—counter to the biasing force of the arms 574 (e.g., in the direction of arrows F in FIG. 5B). The actuator 580 can be formed of plastic, metal, and/or other suitable materials and can be a single integral part or can be formed of discrete parts coupled together.

In the withdrawn position shown in FIGS. 5A and 5B, the arms 574 of the lock members 570 are biased outwardly such that end portions of the arms 574 engage the flange 524 of the barrel 520. Accordingly, the arms 574 inhibit/lock the plunger 110 from advancing farther into the barrel 520 toward the depressed position. To unlock the plunger 510, a user can push the push portion 582 of the actuator to move the push portion 582 at least partially from the first position to the second position (e.g., toward the barrel 520 and the lock members 570). The movement of the push portion 582 drives the arms 584 through the openings 576 in the bodies 572 and over/around the arms 574 of the lock members 570 to move the arms 574 inward toward the longitudinal axis L and out of engagement with the flange 524. The user can then advance the plunger 510 toward the depressed position (FIG. 5C) to expel any contents collected in the barrel 520.

To subsequently withdraw the plunger 510 from the depressed position to the retracted position, the user can first move the actuator 580 to the first position to free the arms 574 of the lock members 570 from the arms 584 of the actuator 580. Accordingly, the arms 574 of the lock members 570 can return to their outwardly-biased position and, in some embodiments, can contact an inner surface of the barrel portion 522. Then, as the plunger 510 is withdrawn through the barrel 520, the arms 574 of the of lock members 570 can spring (i) outward as they are withdrawn from the barrel 520 (e.g., after they past the flange 524) and (ii) into engagement with the flange 524. Accordingly, in one aspect of the present technology, the plunger 510 is automatically locked in position—via the arms 574 of the lock members 570—when the plunger 510 is withdrawn a selected distance.

In some embodiments, the lock members 570 can be configured (e.g., shaped, positioned) such that the syringe 500 automatically locks at a selected position corresponding to a selected volume of the barrel 520. In some embodiments, the selected volume can be about 60 cc or greater. In some embodiments, the syringe 500 can include only one of the lock members 570, or more than two of the lock members 570.

FIG. 6A is a side view of a vacuum-pressure locking syringe (“syringe 600”) in accordance with additional embodiments of the present technology. The syringe 600 is in a depressed position in FIG. 6A. The syringe 600 can include several features generally similar or identical to the syringe 100 described in detail above with reference to FIGS. 1A-3C. In the illustrated embodiment, for example, the syringe 600 includes a plunger 610 slidably positioned within a barrel 620. The barrel 620 is shown as partially transparent in FIG. 6A for clarity. The syringe 600 further includes a base 640 and a lock member 650 operably coupled to the base 640. The lock member 650 includes a lock plate 652 coupled to a button portion 654. The plunger 610 includes a lower edge or surface 611 including a plurality of lock features 613 extending/protruding from the lower surface 611. Each of the lock features 613 can include (i) a ramp portion 615, (ii) a plateau portion 617 extending from the ramp portion 115, and (iii) a stop surface 619 extending from the plateau portion 617.

When the plunger 610 is withdrawn through the barrel 620, the lock features 613 are configured to engage the lock plate 652 to automatically lock the plunger 110 in a withdrawn position corresponding to the position of the lock feature 613. More specifically, a user can withdraw the plunger 610 to a desired location/volume and the lock feature 613 nearest the lock plate 652 can engage the lock plate 652 to automatically lock the plunger 610 in position. To move the syringe 600 from the withdrawn position to the depressed position, the user can (i) actuate the button portion 654 of the lock member 650 to disengage the lock plate 652 from the lock feature 613 to unlock the plunger 610 from the lock member 650 and then (ii) push the plunger 610 relative to the barrel 620. In one aspect of the present technology, the syringe 600 includes multiple ones of the lock features 613 that permit the syringe 600 to be automatically locked at multiple different positions. In the illustrated embodiment, the plunger 610 includes 12 locking features while, in other embodiments, the syringe 600 can include any number of lock features 613 to facilitate locking of the syringe 600 at any desired position/volume. Moreover, the location of the lock features 613 can be selected to provide refined volume and vacuum control.

In the illustrated embodiment, the syringe 600 further includes a locking mechanism 690 that is actuatable to enable/disable the auto-locking functionality of the syringe 600. More specifically, FIGS. 6B and 6C are enlarged side views of the locking mechanism 690 of the syringe 600 in a first position and a second position, respectively, in accordance with embodiments of the present technology. Referring to FIGS. 6B and 6C together, the locking mechanism 690 can include a pin 692 operably coupled to the base 650 via a biasing member 694. In the illustrated embodiment, the pin 692 includes a head portion 693 and an engagement portion 695. The biasing member 694 can be operably coupled between the head portion 693 and the base 640, and the engagement portion 695 can at least partially extend through a channel 696 in the base 640. The lock member 650 can include an opening 698 configured to receive the engagement portion 695 of the pin 692 in the first position shown in FIG. 6B.

In the first position shown in FIG. 6B, the biasing member 694 (e.g., a tension spring) biases the lock pin 692 toward the lock member 650 such that the engagement portion 695 is positioned in the opening 698. Referring to FIGS. 6A and 6B together, in the first position the lock member 650 is partially depressed such that the lock features 613 are free to slide past the lock plate 652 without engaging the lock plate 652. Accordingly, the plunger 610 can be withdrawn/depressed through the barrel 620 without the lock features 613 automatically locking the syringe 600—and thus the syringe 600 functions similar to a conventional syringe when the locking mechanism 690 is in the first position. To enable to the auto-locking functionality of the syringe 600, a user can pull the pin 692 (e.g., the head portion 693) away from the lock member 650 (e.g., against the biasing force of the biasing member 694) to withdraw the engagement portion 695 from the opening 698 and disengage the engagement portion 695 from the lock member 650. Accordingly, in the second position, the lock member 650 is operable to engage the lock features 613 to auto-lock the syringe 600 (e.g., the lock member 650 can be biased towards as described with reference to FIGS. 3A-4). In other embodiments, the pin 692 can be operably coupled to the lock member 650 and configured to engage the base 640. In some embodiments, the syringe 100 and/or the syringe 500 described in detail with reference to FIGS. 1A-5C can include an identical or similar locking mechanism for enabling/disabling the autolocking functionality.

### II. SELECTED EMBODIMENTS OF CLOT TREATMENT SYSTEMS

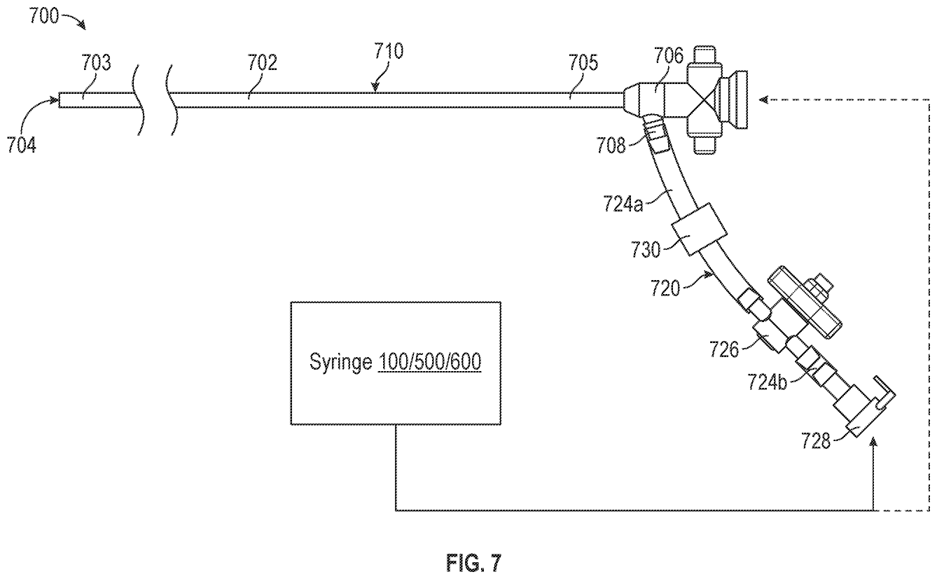

FIG. 7 is a partially schematic side view of a clot treatment or clot removal system comprising an aspiration assembly 700 (“assembly 700”) incorporating the syringe 100, the syringe 500, and/or the syringe 600 in accordance with embodiments of the present technology. In the illustrated embodiment, the assembly 700 includes a catheter subsystem 710 fluidly coupled to a tubing subsystem 720. In general, the assembly 700 (i) can include features generally similar or identical to those of the aspiration assemblies described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter subsystem 710 includes a catheter 702 (e.g., an aspiration catheter) comprising an elongated shaft defining a lumen 704 and having a distal portion 703 and a proximal portion 705. The catheter subsystem 710 further includes a valve 706 that can be integral with or coupled to the proximal portion 705 of the catheter 702. In some embodiments, the valve is a hemostasis valve that is configured to maintain hemostasis during a clot removal procedure by preventing fluid flow in the proximal direction through the valve 706 as various components such as delivery sheaths, pull members, guidewires, interventional devices, other aspiration catheters, and so on are inserted through the valve 706 to be delivered through the catheter 702 to a treatment site in a blood vessel. The valve 706 includes a branch or side port 708 configured to fluidly couple the lumen 704 of the catheter 702 to the tubing subsystem 720. In some embodiments, the valve 706 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

The tubing subsystem 720 fluidly couples the catheter subsystem 710 to the syringe 100, the syringe 500, and/or the syringe 600 (collectively the “syringe 100/500/600”). More specifically, the tubing subsystem 720 can include one or more tubing sections 724 (individually labeled as a first tubing section 724a and a second tubing section 724b), at least one fluid control device 726 (e.g., a valve), and at least one connector 728 (e.g., a Toomey tip connector) for fluidly coupling the tubing subsystem 720 to the syringe 100/500/600 and/or other suitable components. More specifically, in the illustrated embodiment the fluid control device 726 is a stopcock that is fluidly coupled to (i) the side port 708 of the valve 706 via the first tubing section 724a and (ii) the connector 728 via the second tubing section 724b.

The fluid control device 726 is externally operable by a user to regulate the flow of fluid therethrough and, specifically, from the lumen 704 of the catheter 702 to the syringe 100/500. In some embodiments, the connector 728 is a quick-release connector (e.g., a quick disconnect fitting) that enables rapid coupling/decoupling of the catheter 702 and the fluid control device 726 to/from the syringe 100/500/600.

With reference to FIGS. 1A-7 together, the syringe 100/500/600 is configured to generate (e.g., form, create, charge, build-up) a vacuum (e.g., negative relative pressure) and store the vacuum for subsequent application to the catheter subsystem 710. For example, during operation of the assembly 700, a user can first close the fluid control device 726 before (i) withdrawing the plunger 110 of the syringe 100 until the plunger 110 automatically locks to build up vacuum pressure within the barrel 120 of the syringe 100, (ii) withdrawing the plunger 510 of the syringe 500 until the plunger 510 automatically locks to build up vacuum pressure within the barrel 520 of the syringe 500, and/or (iii) withdrawing the plunger 610 of the syringe 600 until the plunger 610 automatically locks at a location corresponding to one of the lock features 613. In this manner, a vacuum is charged within the syringe 100/500/600 (e.g., a negative pressure is maintained) before the syringe 100/500/600 is fluidly connected to the catheter subsystem 710. To aspirate the lumen 704 of the catheter 702, the user can open the fluid control device 726 to fluidly connect the syringe 100/500/600 to the catheter subsystem 710 and thereby apply or release the vacuum stored in the syringe 100/500/600 to the lumen 704 of the catheter 702.

Opening of the fluid control device 726 instantaneously or nearly instantaneously applies the stored vacuum pressure to the tubing subsystem 720 and the catheter 702, thereby generating a suction pulse throughout the catheter 702. In particular, the suction is applied at the distal portion 703 of the catheter 702. In one aspect of the present technology, pre-charging or storing the vacuum in the syringe 100/500/600 before applying the vacuum to the lumen 704 of the catheter 702 is expected to generate greater suction forces and corresponding fluid flow velocities at and/or near the distal portion 703 of the catheter 702 compared to simply activating the syringe 100/500/600 while it is fluidly connected to the catheter 702. When the distal portion 703 is intravascularly positioned near clot material within a patient (e.g., a human patient), the suction forces generated by application of the stored vacuum can be used to aspirate or otherwise remove clot material from within a blood vessel of the patient.

### III. SELECTED EMBODIMENTS OF VACUUM INDICATORS

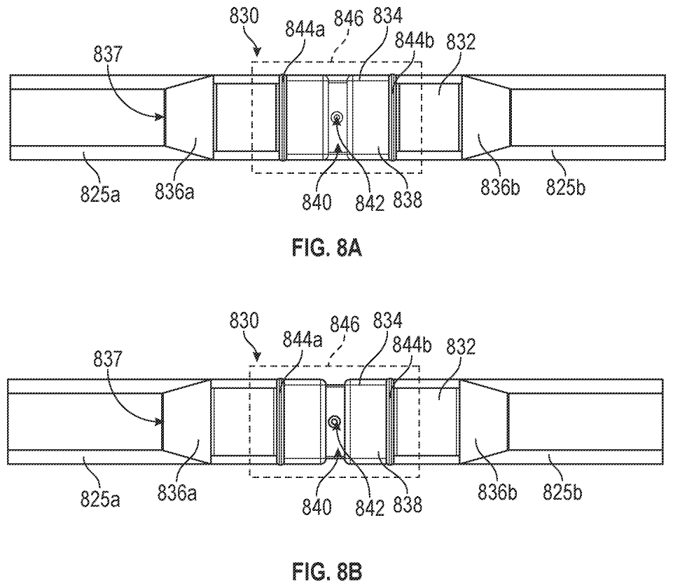

Referring to FIG. 7, in some embodiments the assembly 700 can include a vacuum indicator 730 (shown schematically) operably coupled to the tubing subsystem 720. As described in greater detail below with reference to FIGS. 8A and 8B, the vacuum indicator 730 can provide an indication, such as a visual indication, of whether the assembly 700 is under vacuum pressure. In the illustrated embodiment, the vacuum indicator 730 is operably coupled to the tubing subsystem 720 between the fluid control device 726 and the valve 706 (e.g., to the first tubing section 724a, between separate portions of the first tubing section 724a). In other embodiments, the vacuum indicator 730 can be operably coupled to the tubing subsystem 720 between the fluid control device 726 and the connector 728 (e.g., to the second tubing section 724b, between separate portions of the second tubing section 724b).

FIGS. 8A and 8B are enlarged, partially-schematic side views of the vacuum indicator 730 in a vacuum-off position and a vacuum-on position, respectively, in accordance with embodiments of the present technology. Referring to FIGS. 8A and 8B together, the vacuum indicator 730 includes a body 832 and a flexible member 834 operably coupled to the body 832. The flexible member 834 is shown as partially transparent in FIGS. 8A and 8B for clarity. In some embodiments, the body 832 is an integral or one-piece member of plastic, metal, or another suitably rigid material. The body 832 can include a first connector portion 836a (e.g., a first barb), a second connector portion 836b (e.g., a second barb), and a central portion 838 extending between the first and second connector portions 836a, b. The body 832 can further define a lumen 837 extending therethrough between the first and second connector portions 836a, b. The first connector portion 836a can be coupled to a first tube 825a and the second connector portion 836b can be coupled to a second tube 825b. In some embodiments, the first and second tubes 825a, b can together form the first tubing section 724a (FIG. 7) such that the vacuum indicator 730 is fluidly coupled between the fluid control device 726 and the valve 706.

In the illustrated embodiment, the central portion 838 of the body 832 includes/defines (i) a recess 840 extending circumferentially thereabout (ii) and an opening 842 positioned in the recess 840 and extending through the central portion 838 to the lumen 837. In some embodiments, the recess 840 can have a different shape and/or can extend only partially around the central portion 838, and/or the body 832 can include more than one of the openings 842 positioned in the recess 840. The flexible member 834 can be a thin compliant tube that is positioned over the recess 840 and the opening 842. In some embodiments, opposing end portions of the flexible member 834 are secured to the body 832 via a first attachment member 844a and a second attachment member 844b. The first and second attachment members 844a, b can comprise adhesives, welds, fasteners, collars, or other components that mechanically secure the flexible member 834 over the recess 840 such that the flexible member 834 forms a seal over the recess 840. In some embodiments, the flexible member 834 can have a thickness of between about 0.005 inch to about 0.100 inch.

With additional reference to FIG. 7, when the assembly 700 is not under vacuum, the flexible member 834 is positioned over the recess 840 in a relaxed state such that the flexible member 834 has a generally cylindrical shape as shown in FIG. 8A. When a vacuum is generated in the assembly 700, the vacuum is applied to the flexible member 834 via the opening 842 and pulls/sucks the flexible member 834 into the recess 840 as shown in FIG. 8B. The deformation of the flexible member 834 provides a visual indication to a user of the assembly 700 that the assembly 700 is under vacuum. When the vacuum is released, the flexible member 834 returns/rebounds to the vacuum-off position shown in FIG. 8A to provide a visual indication that the assembly 700 is not under vacuum.

In some embodiments, the thickness of the flexible member 834, the size of the opening 842, and/or the number of openings 842 can be selected such that the flexible member 834 deforms to the vacuum-on position (FIG. 8B) and returns to the vacuum-off position (FIG. 8A) at a selected level of vacuum. For example, making the flexible member 834 thinner will cause it to indent under less vacuum while making the flexible member 834 thicker will require more vacuum to indent. Likewise, a thinner flexible member 834 will be less likely to rebound to the vacuum-off position (FIG. 8A) and therefore require more pressure (e.g., positive pressure) to rebound, while a thicker flexible member 834 will rebound with little to no pressure.

In some embodiments, the vacuum indicator 730 can include a housing 846 (shown schematically) at least partially surrounding the flexible member 834. The housing 846 can be formed of a rigid material (e.g., plastic, metal) and can be coupled to the body 832. In some embodiments, the housing 846 is configured to inhibit or even prevent excessive deformation of the flexible member 834 when the assembly 700 is under positive pressure. For example, when positive pressure is applied to the assembly 700, the flexible member 834 can flex outwardly away from the body 832 and contact the housing 846. The housing 846 can therefore inhibit the flexible member 834 from flexing further outwardly, which could damage or rupture the flexible member 834.

In some aspects of the present technology, the vacuum indicator 730 can aid the user during a clot removal procedure on a patient using the assembly 700. Sometimes, for example, after applying the vacuum stored in the syringe 100/500/600 to the lumen 704 of the catheter 702, clot material can at least partially clog the distal portion 703 of the catheter 702—causing the assembly 700 to cavitate. In such instances, it is often desirable to remove the assembly 700 from the patient to thereby remove the clot material clogging and/or attached to the distal portion 703 of the catheter 702. However, user error and/or leakage of the assembly 700 (e.g., out of the valve 706) can dissipate the vacuum in the assembly 700, thereby releasing and/or decreasing the force holding the clot material to the catheter 702. The vacuum indicator 730 can provide a fast and easy visual indication to the user of whether the assembly 700 is maintaining vacuum. Thus, based on the position of the vacuum indicator 730, the user can determine how to proceed with the procedure—for example, whether to continue withdrawing the assembly 700 from the patient.

In some aspects of the present technology, the vacuum indicator 730 is a passive device that adds no or very little volume to the flow path of the assembly 700. Adding volume to the flow path of the assembly 700 can reduce the amount of aspirational force that can be generated at the distal portion 703 of the catheter 702. Accordingly, the vacuum indicator 730 can provide an indication of vacuum (e.g., on or off) without negatively affecting the clot removal capabilities of the assembly 700. In contrast, for example, a vacuum gauge coupled along the flow path of the assembly 702 would increase the volume thereof and reduce the aspirational force of the assembly 700. Moreover, the recess 840 can have a relatively small volume such that the vacuum indicator 730 is not prone to clogging during use of the assembly 700 (e.g., clogging with blood clots during a thrombectomy procedure using the assembly 700). In contrast, a vacuum gauge or similar mechanism would be more prone to clogging, possibly rendering them non-functional.

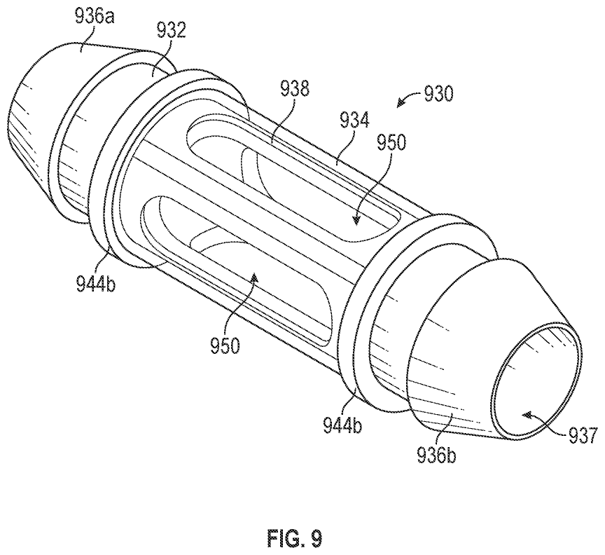

FIG. 9 is a perspective view of a vacuum indicator 930 in a vacuum-off position in accordance with additional embodiments of the present technology. The vacuum indicator 930 can include some features, functions, and/or advantages that are generally similar or identical to the vacuum indicator 730 described in detail above with reference to FIGS. 8A and 8B. For example, in the illustrated embodiment the vacuum indicator 930 includes a body 932 and a flexible member 934 operably coupled to the body 932. The flexible member 934 is shown as partially transparent in FIG. 9 for clarity. The body 932 can include a first connector portion 936a (e.g., a first barb), a second connector portion 936b (e.g., a second barb), and a central portion 938 extending between the first and second connector portions 936a, b. The body 932 can further define a lumen 937 extending therethrough between the first and second connector portions 936a, b. The first and second connector portions 936a, b can be coupled to, for example, various tubes of the first tubing section 724a and/or the second tubing section 724b (FIG. 7).

In the illustrated embodiment, the central portion 938 of the body 932 includes/defines a plurality of longitudinal openings 950 extending through the central portion 938 to the lumen 937. In some embodiments, the openings 950 can be equally spaced about the body 932. In other embodiments, the central portion 938 can include more or fewer of the openings 950 and/or the openings 950 can be positioned differently about the central portion 938. The flexible member 934 can be a thin compliant tube that is sealingly secured to the body 932 over the openings 950 via a first attachment member 944a and a second attachment member 944b.

With additional reference to FIG. 7, when the assembly 700 is not under vacuum, the flexible member 934 is positioned over the openings 950 in a relaxed state such that the flexible member 934 has a generally cylindrical shape as shown in FIG. 9. When a vacuum is generated in the assembly 700, the vacuum is applied to the flexible member 934 via the openings 950 and pulls the flexible member 934 at least partially into one or more of the openings 950. The deformation of the flexible member 934 provides a visual indication to a user of the assembly 700 that the assembly 700 is under vacuum. When the vacuum is released, the flexible member 934 can return to the position vacuum-off shown in FIG. 9 to provide a visual indication that the assembly 700 is not under vacuum. In some embodiments, the vacuum indicator 930 can include a housing (not shown) at least partially surrounding the flexible member 934 and configured to inhibit or even prevent excessive deformation of the flexible member 934 when the assembly 700 is under positive pressure.

FIGS. 10A and 10B are perspective views of a vacuum indicator 1030 in a vacuum-off position and a vacuum-on position, respectively, in accordance with additional embodiments of the present technology. The vacuum indicator 1030 can include some features, functions, and/or advantages that are generally similar or identical to the vacuum indicator 730 and/or the vacuum indicator 930 described in detail above with reference to FIGS. 8A-9. For example, referring to FIGS. 10A and 10B together, the vacuum indicator 930 includes a flexible member 934 fluidly coupled to a tube 1025. In some embodiments, the tube 925 is a portion of the first tubing section 724a or the second tubing section 724b (FIG. 7).

In the illustrated embodiment, however, the flexible member 1034 is directly attached to the tube 1025 over a hole or opening 1027 (obscured in FIG. 10A and shown schematically) in the tube 1025. In some embodiments, opposing end portions of the flexible member 1034 are secured to the tube 1029 via a first attachment member 1044a and a second attachment member 1044b. The first and second attachment members 1044a, b can be clamps, collars, or other mechanical components that mechanically secure the flexible member 1034 over the opening 1027 such that the flexible member 1034 forms a seal over the opening 1027. The first and second attachment members 1044a, b can be secured to the tube 1025 via compression, adhesives, fasteners, and/or other suitable means of connection. In some embodiments, the opening 1027 can have a diameter of about 0.100 inch to about 0.300 inch.

With additional reference to FIG. 7, when the assembly 700 is not under vacuum, the flexible member 1034 is positioned over the opening 1027 in a relaxed state such that the flexible member 1034 has a generally cylindrical shape as shown in FIG. 10A. When a vacuum is generated in the assembly 700, the vacuum is applied to the flexible member 1034 via the opening 1027 and pulls the flexible member 1034 into the opening 1027 as shown in FIG. 10B. The deformation of the flexible member 1034 provides a visual indication to a user of the assembly 700 that the assembly is under vacuum. When the vacuum is released, the flexible member 1034 returns to the vacuum-off position shown in FIG. 10A to provide a visual indication that the assembly 700 is not under vacuum.

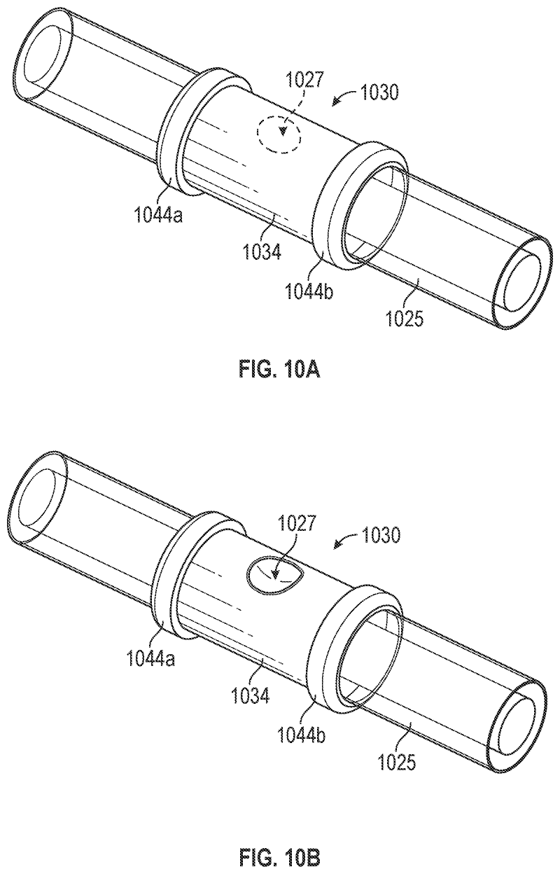

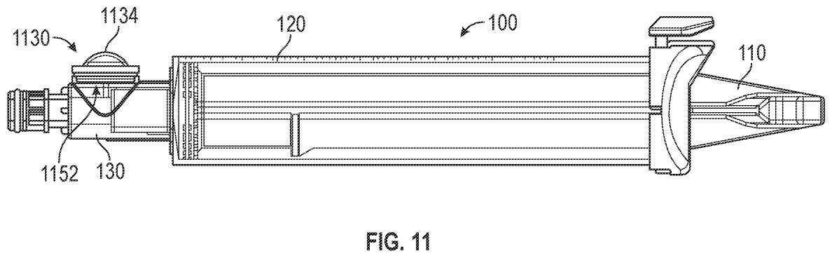

In some embodiments, a syringe configured in accordance with the present technology can include a vacuum indicator integrated therewith. For example, FIG. 11 is a side-view of the syringe 100 of FIGS. 1A and 1B including a vacuum indicator 1130 formed in the adaptor 130 in accordance with embodiments of the present technology. In the illustrated embodiment, the vacuum indicator 1130 includes a flexible member 1134 sealingly positioned over an opening 1152 in the adaptor 130. The flexible member 1134 can be formed from a compliant material, such as silicone, and can have a dome-like shape in a vacuum-off position shown in FIG. 11. With additional reference to FIG. 7, when the syringe 100 is coupled to the system 700 (e.g., to the connector 728) and the plunger 110 is drawn to pull a vacuum in the barrel 120, the vacuum is applied to the flexible member 1134 via the opening 1152 and pulls the flexible member 1134 into/toward the opening 1152. For example, the flexible member 1134 can invert when the assembly 700 is under vacuum. The inversion/deformation of the flexible member 1134 provides a visual indication to a user of the assembly 700 that the assembly 700 is under vacuum. When the vacuum is released, the flexible member 1134 can passively return to the vacuum-off position shown in FIG. 11 to provide a visual indication that the assembly 700 is not under vacuum.

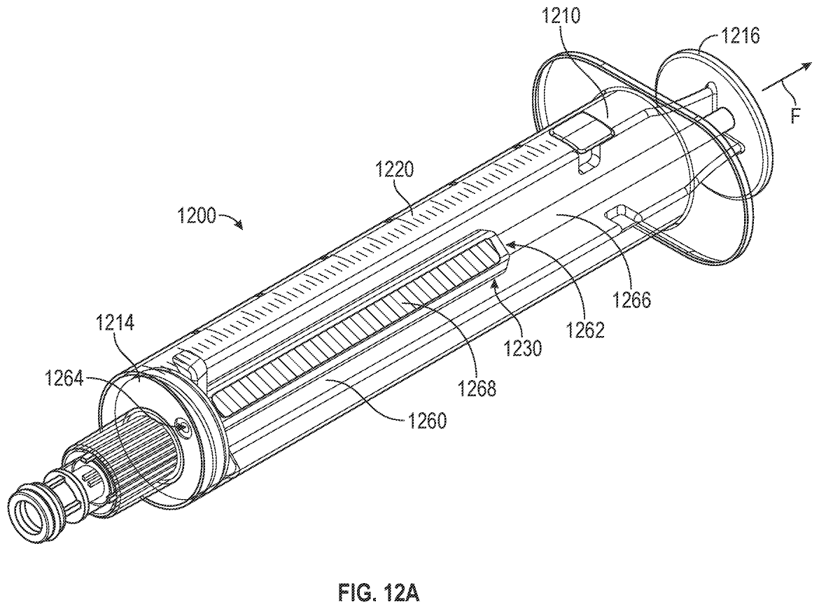

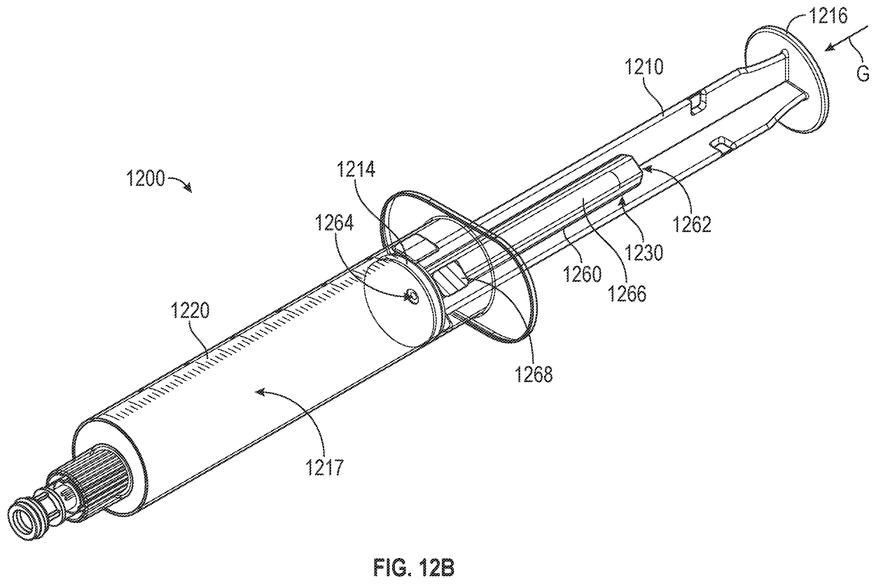

FIGS. 12A and 12B are perspective views of a syringe 1200 including a vacuum indicator 1230 in accordance with additional embodiments of the present technology. The syringe 1200 is in a withdrawn position in FIG. 12B. Referring to FIGS. 12A and 12B together, the syringe 1200 includes a plunger 1210 slidably positioned within a barrel 1220. The barrel 1220 is shown as partially transparent in FIG. 1B for clarity. The plunger 1210 includes a flange 1214 configured to sealingly engage an inner surface of the barrel 1220 to define a sealed volume 1217 (FIG. 12B: e.g., of negative/vacuum pressure) within the barrel 1220.

In the illustrated embodiment, the vacuum indicator 1230 includes a housing 1260 (shown as partially transparent in FIGS. 12A and 12B) coupled to the plunger 110 and defining a lumen 1262. The flange 1214 can include a through-hole 1264 fluidly coupling the lumen 1262 of the housing 1260 to the sealed volume 1217 within the barrel 1220. The vacuum indicator 1230 can further include an indication member 1266 slidably positioned within the lumen 1262. The indication member 1266 can be an elongate member and, in some embodiments, can have a color (e.g., a relatively bright color) that contrasts with a color of the housing 1260. In some embodiments, the indication member 1266 is configured to sealingly engage the housing 1260. For example, the housing 1260 can include one or more sealing members (e.g., O-rings; not shown) on an inner surface thereof and/or the indication member 1266 can include one or more sealing members (e.g. O-rings; not shown) on an outer surface thereof to provide a dynamic seal between the housing 1260 and the indication member 1266. In the illustrated embodiment, the indication member 1266 is operably coupled to the flange 1214 via a biasing member 1268 that extends through the lumen 1262. The biasing member 1268 (e.g., a compression spring) is configured to bias the indication member 1266 through the lumen 1262 in a direction away from the flange 1214 (e.g., as indicated by arrow F in FIG. 12A).

With additional reference to FIG. 7, when the syringe 1200 is coupled to the system 700 (e.g., to the connector 728) and the plunger 1210 is drawn to pull a vacuum in the sealed volume 1217 of the barrel 1220, the vacuum is applied to the indication member 1266 via the through-hole 1264 in the flange 1214 and the lumen 1262 of the housing 1260. The vacuum pulls the indication member 1266 through the lumen 1262 against the biasing force of the biasing member 1268 as indicated by arrow G in FIG. 12B. The positioning of the indication member 1266 relative to the housing 1260 can provide a visual indication to a user of the assembly 700 that the assembly 700 is under vacuum. In some embodiments, for example, when the plunger 1210 is fully withdrawn, the indication member 1266 can be positioned entirely within the lumen 1262 and thus obscured by the housing 1260 as shown in FIG. 12B. When the vacuum is released, the biasing member 1268 drives the indication member 1266 through the lumen 1262 in the direction of arrow F at least partially outside the housing 1260 (e.g., as shown in FIG. 12A) to provide a visual indication that the assembly 700 is not under vacuum. In some aspects of the present technology, the position of the indication member 1266 relative to the housing 1260 can provide an indication of a varying level of vacuum in the assembly 700 (e.g., in contrast to a binary on/off indication).

In some embodiments, the plunger 1210 includes a grip portion 1216 configured to engage the indication member 1266, as shown in FIG. 12A, to inhibit further movement of the indication member 1266 in the direction of arrow F when the assembly 700 is under positive pressure. In some embodiments, any of the vacuum-pressure locking syringes 100/500/600 described in detail above with reference to FIGS. 1A-6C can include the vacuum indicator 1230.

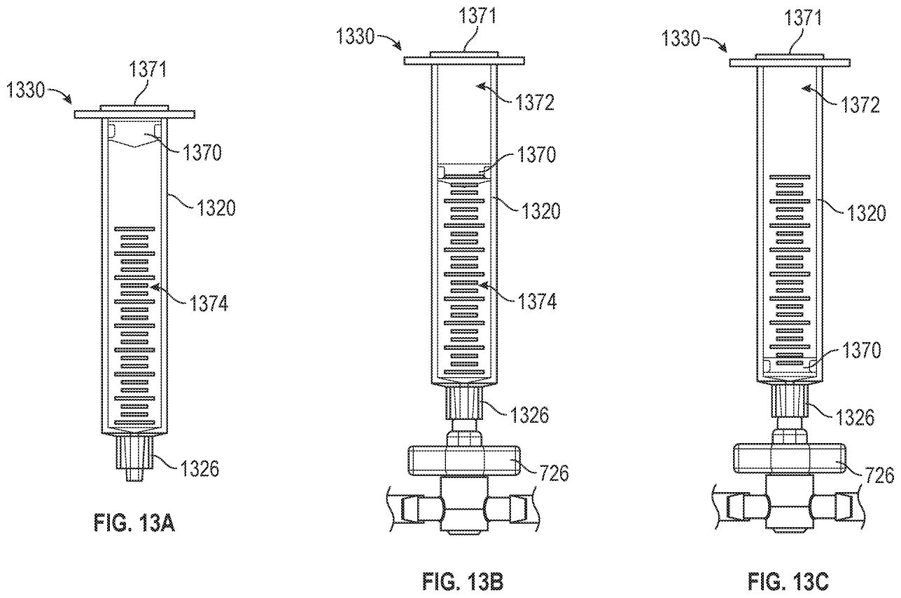

FIGS. 13A-13C are side views of a vacuum indication device or vacuum indicator 1330 in a vacuum-off position, a partial-vacuum position, and a full-vacuum position, respectively, in accordance with additional embodiments of the present technology. Referring to FIGS. 13A-13C together, the vacuum indicator 1330 includes a sealing indicator 1370 slidably positioned within a barrel 1320. The barrel 1320 includes a cap 1371 (e.g., a sealed cap), and a tip 1326 (e.g., a Luer connector) that can be directly coupled to another device or system, such as the fluid control device 726 (e.g., stopcock) of FIG. 7, as shown in FIGS. 13B and 13C.

In some embodiments, the sealing indicator 1370 is configured to sealingly engage the barrel 1320 to define a first volume or chamber 1372 and a second volume or chamber 1374 within the barrel 1320. For example, the sealing indicator 1370 can include one or more sealing members (e.g. O-rings; not shown) on an outer surface thereof to provide a dynamic seal between the barrel 1320 and the sealing indicator 1370. In some embodiments, the one or more sealing members can engage the barrel 1320 with low friction to facilitate sliding movement of the sealing indicator 1370 within the barrel 1320. The first chamber 1372 can be a sealed volume with no outlet, while the second chamber 1374 can be open to the tip 1326.

With additional reference to FIG. 7, when the assembly 700 is not under vacuum pressure, the sealing indicator 1370 can be positioned near an upper portion of the barrel 1320 proximate the cap 1371. When the syringe 100/500/600 is used to generate a vacuum in the assembly 700, vacuum pressure is generated in the second chamber 1374. The vacuum pressure in the second chamber 1374 pulls the sealing indicator 1370 downward toward the tip 1326 of the barrel 1320 as shown in FIG. 13B. As the sealing indicator 1370 moves downward away from the cap 1371, the volume of the first chamber 1372 increases, thereby generating vacuum pressure in the first chamber 1372. The vacuum pressure generated in the first chamber 1372 provides a resistive force that acts against the vacuum force in the second chamber 1374 to urge the sealing indicator 1370 upward toward the cap 1371. The vacuum force in second chamber 1374 must overcome the resistive vacuum force in first chamber 1372 to cause the sealing indicator 1370 to travel downward through the barrel 1320. In some embodiments, the vacuum indicator 1370 is configured (e.g., shaped, sized) such that the sealing indicator 1370 abuts the tip 1326 of the barrel 1320 in the full-vacuum position, as shown in FIG. 13C, to indicate that a maximum vacuum level has been generated in the assembly 700. If vacuum force dissipates or is lost in the assembly 700, the resistive vacuum force in the first chamber 1372 can return the sealing indicator 1370 toward the vacuum-off position shown in FIG. 13A.

In general, the opposing vacuum forces in the first and second volumes 1372, 1374, along with the friction forces between the barrel 1320 and the sealing indicator 1370, dictate the travel speed and location of the sealing indicator 1370 along the barrel 1320. In some aspects of the present technology, the vacuum indicator 1330 can provide a continuous resolution of the vacuum level at higher vacuum levels (e.g., greater than 25 inHg). That is, the position of the sealing indicator 1370 in the barrel 1320 can indicate a specific level of vacuum in the assembly 700.

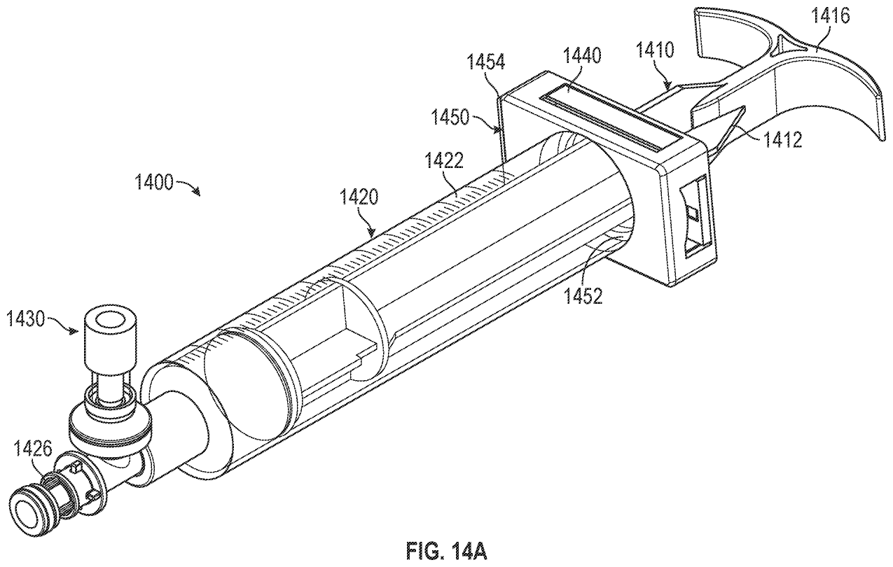

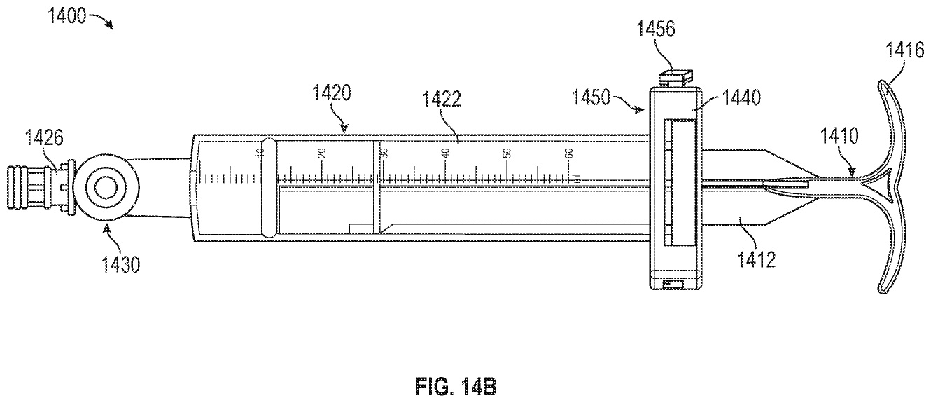

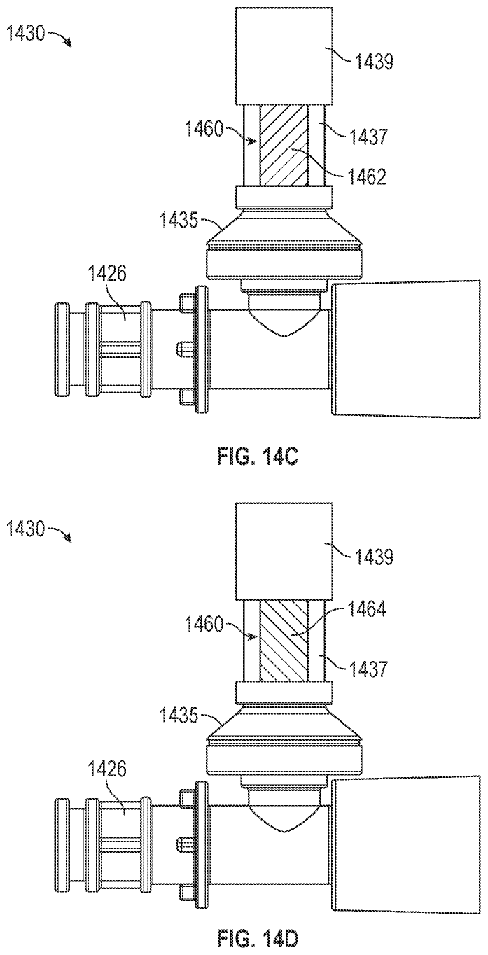

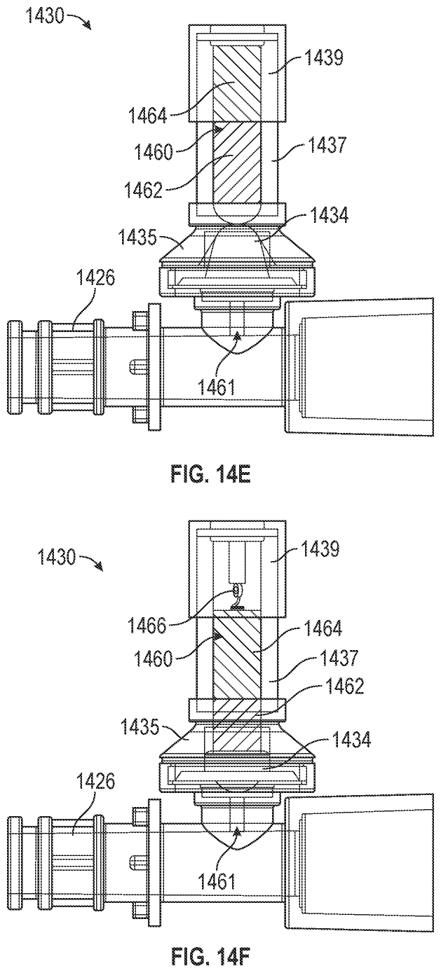

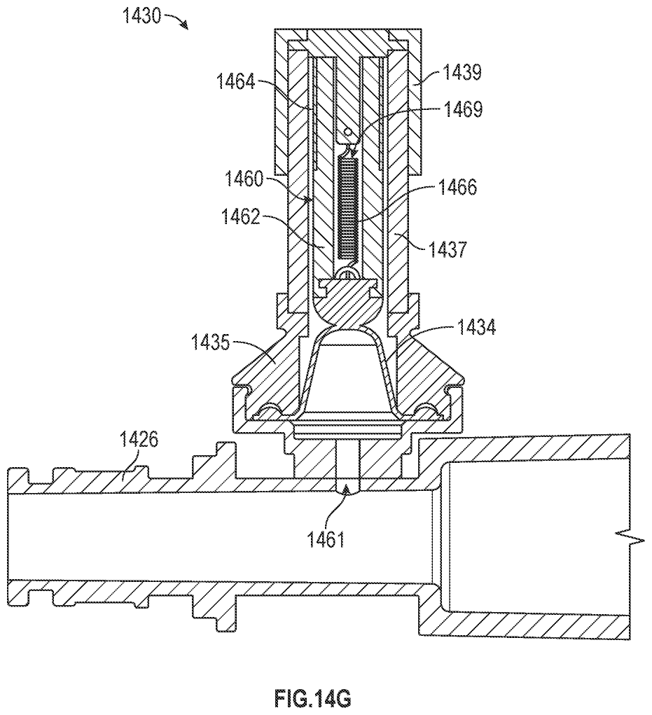

FIGS. 14A and 14B are an isometric view and a top view of a syringe 1400 in accordance with additional embodiments of the present technology. The syringe 1400 is in a depressed/advanced position in FIGS. 14A and 14B. Referring to FIGS. 14A and 14B together, the syringe 1400 can include several features generally similar or identical to the syringe 100 described in detail above with reference to FIGS. 1A-4, and can be coupled to the assembly 700 of FIG. 7 for use in a clot removal procedure. In the illustrated embodiment, for example, the syringe 1400 includes a plunger 1410 configured to be slidably positioned within a barrel 1420. The barrel 1420 is shown as partially transparent in FIGS. 14A and 14B for clarity. The barrel 1420 can include a barrel portion 1422 and a tip 1426. The plunger 1410 can include a shaft 1412 and a grip portion 1416. The syringe 1400 further includes a base 1440 and a lock member 1450 having a lock plate 1452 (obscured in FIG. 14B and partially obscured in FIG. 14A) and a button portion 1454. The lock member 1450 is configured to automatically engage the base 1440 when the plunger 1410 is withdrawn to automatically lock the plunger 1410 in a withdrawn position, as described in detail above. The button portion 1454 can be actuatable (e.g., depressible) by a user to release the lock plate 1452 from the base 1440 to release the plunger 1410 to, for example, permit the plunger 1410 to move through the barrel 1420 to expel any contents (e.g., bodily fluids such as blood and clot material) collected in the barrel 1420 from the tip 1426. In the illustrated embodiment, however, the button portion 1454 extends generally parallel to the grip portion 1416 of the plunger 1410.

Moreover, in the illustrated embodiment the syringe 1400 further includes a vacuum indicator 1430 formed in and/or coupled to the tip 1426. FIGS. 14C and 14D are enlarged side views of the vacuum indicator 1430 in a “vacuum-off” and a “vacuum-on” position, respectively, in accordance with embodiments of the present technology. FIGS. 14E and 14F are enlarged partially transparent side views of the vacuum indicator 1430 in the vacuum-off and the vacuum-on position, respectively, in accordance with embodiments of the present technology. FIG. 14G is an enlarged side cross-sectional view of the vacuum indicator in the vacuum-off position in accordance with embodiments of the present technology. Referring to FIGS. 14E-14G together, the vacuum indicator 1430 can include a flexible member 1434 (e.g., a diaphragm, a rolling diaphragm) sealingly positioned over an opening 1461 in the tip 1426. The flexible member 1434 can be formed from a compliant material, such as silicone.

Referring to FIGS. 14C-14G together, in the illustrated embodiment the vacuum indicator 1430 further includes (i) a non-transparent (e.g., opaque) bonnet or base 1435 positioned around and/or coupled to the flexible member 1434, (ii) a transparent tube 1437 coupled to and extending from the base 1435, and (iii) a non-transparent cap 1439 coupled to and positioned above the tube 1437. The base 1435, the tube 1437, and/or the cap 1439 can be integrally formed or can be separate components coupled together. The vacuum indicator 1430 can further include an indicator 1460 movably positioned within the tube 1437 and the cap 1439. The indicator 1460 can include a first indicator region 1462 and a second indicator region 1464 each having at last one different visual characteristic (e.g., color, pattern, size, thickness, shape) from one another. For example, in some embodiments the first indicator region 1462 can be a first color (e.g., red) while the second indicator region 1464 can be a different second color (e.g., green). In some embodiments, a first end portion of the indicator 1460 (e.g., a lower end of the first indicator region 1462) can be coupled to the flexible member 1434 and a second end portion of the indicator 1460 (e.g., an upper end of the second indicator region 1464) can be coupled to the cap 1439 via a biasing member 1466 (FIGS. 14F and 14G), such as a spring. As best seen in FIG. 14G, in some embodiments the biasing member 1466 can extend at least partially through an internal channel 1469 of the indicator 1460.

When the tip 1426 of the syringe 1400 is not under vacuum, the vacuum indicator 1430 is in the vacuum-off position in which the biasing member 1466 biases the indicator 1460 toward the cap 1439 such that the first indicator region 1462 is positioned adjacent the transparent tube 1437—and thus visible to the user—as shown in FIGS. 14C and 14E. At the same time, the second indicator region 1464 is obscured by the non-transparent cap 1439 in the vacuum-off position. When the tip 1426 of the syringe 1400 is under vacuum, the vacuum indicator 1430 is in the vacuum-on position in which the vacuum pulls the flexible member 1434 into/toward the opening 1461, thereby pulling the indicator 1460 toward the opening 1461 against the biasing force of the biasing member 1466 such that the second indicator region 1464 is positioned adjacent the transparent tube 1437—and thus visible to the user—as shown in FIGS. 14D and 14F. At the same time, the first indicator region 1462 is obscured by the non-transparent base 1435 in the vacuum-on position. In this manner, the first and second indicator regions 1462, 1464 are configured to provide a visual indication to the user of whether the syringe 1400 (and the coupled assembly 700 of FIG. 7) is under vacuum or not.

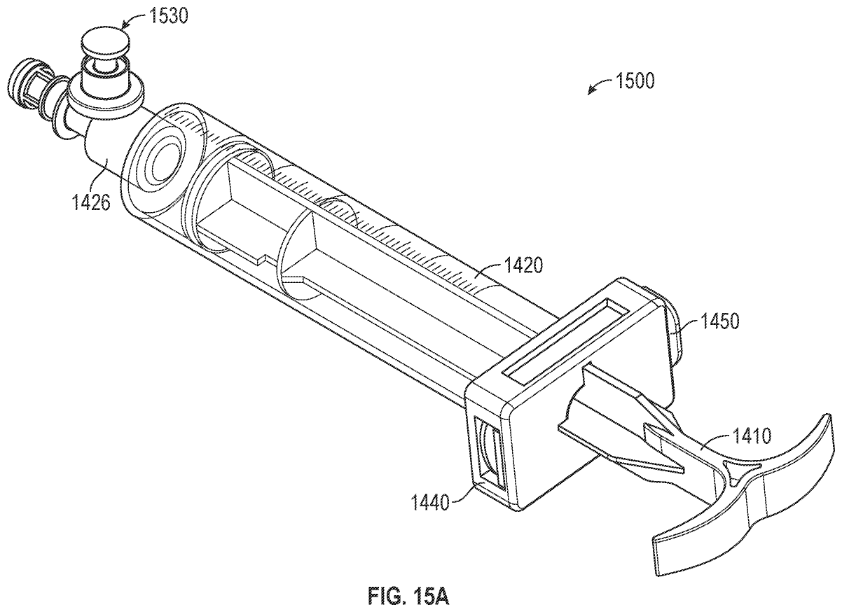

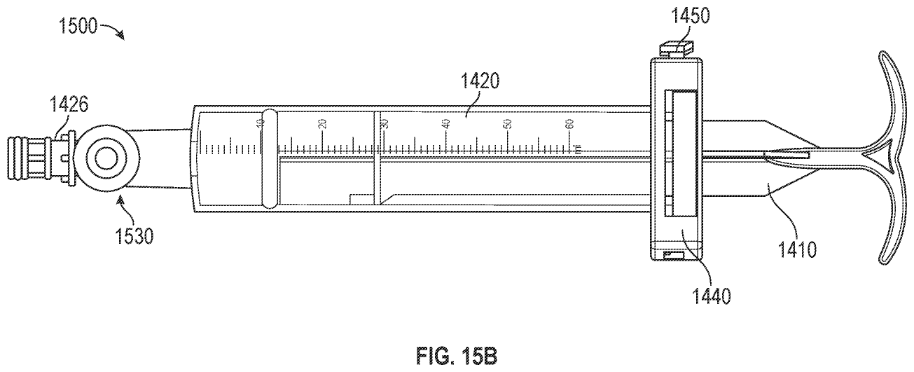

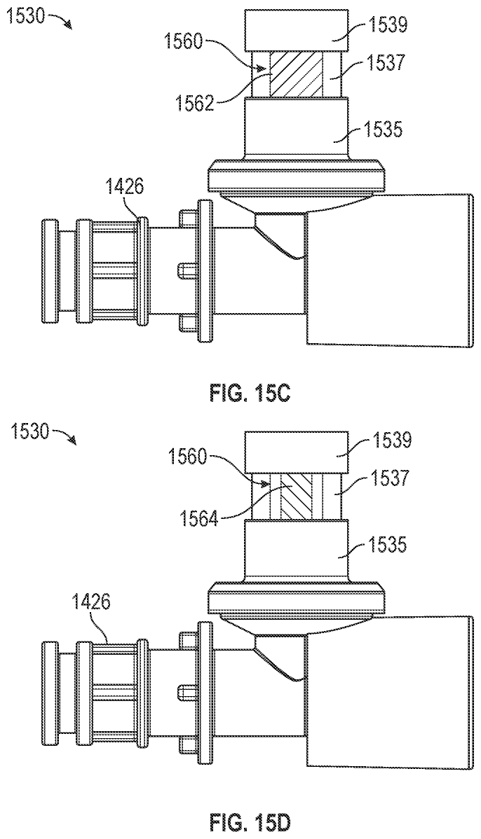

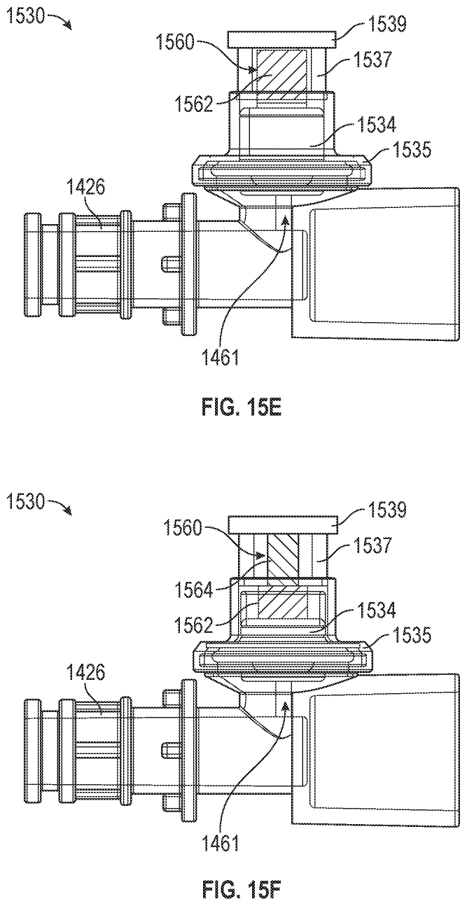

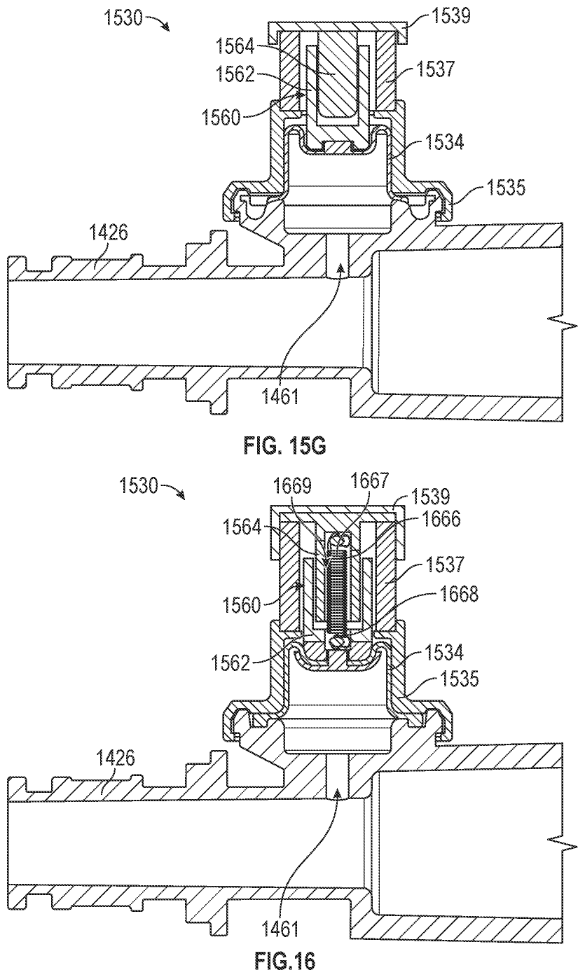

FIGS. 15A and 15B are an isometric view and a side view of a syringe 1500 in accordance with additional embodiments of the present technology. The syringe 1500 is in a depressed/advanced position in FIGS. 15A and 15B. Referring to FIGS. 15A and 15B together, in some embodiments the syringe 1500 can be generally identical to the syringe 1400 described in detail above with reference to FIGS. 14A and 14B (e.g., including the plunger 1410, the barrel 1420, the 1426, the base 1440, and the lock member 1450). In the illustrated embodiment however, the syringe 1500 includes a different vacuum indicator 1530 formed in and/or coupled to the tip 1426.

FIGS. 15C and 15D are enlarged side views of the vacuum indicator 1530 in a “vacuum-off” and a “vacuum-on” position, respectively, in accordance with embodiments of the present technology. FIGS. 15E and 15F are enlarged partially transparent side views of the vacuum indicator 1530 in the vacuum-off and the vacuum-on position, respectively, in accordance with embodiments of the present technology. FIG. 15G is an enlarged side cross-sectional view of the vacuum indicator in the vacuum-off position in accordance with embodiments of the present technology. The vacuum indicator 1530 can include several features that are at least generally similar in structure and function, or identical in structure and function, to the corresponding features of the vacuum indicator 1430 described in detail above with reference to FIGS. 14C-14G, and can operate in a generally similar or identical manner to the vacuum indicator 1430.

For example, referring to FIGS. 15E-15G together, the vacuum indicator 1530 can include a flexible member 1534 (e.g., a diaphragm, a rolling diaphragm) sealingly positioned over the opening 1461 in the tip 1426. The flexible member 1534 can be biased to a first (e.g., preconvoluted) position in the vacuum-off position and can be pulled downward toward the opening 1461 to a second position in the vacuum-on position. Referring to FIGS. 15C-15G together, the vacuum indicator 1530 can further include (i) a non-transparent (e.g., opaque) base 1535 positioned around and/or coupled to the flexible member 1534, (ii) a transparent tube 1537 coupled to and extending from the base 1535, and (iii) a non-transparent cap 1539 coupled to and positioned above the tube 1537. The base 1535, the tube 1537, and/or the cap 1539 can be integrally formed or can be separate components coupled together. The vacuum indicator 1530 further includes an indicator 1560 positioned within the tube 1537 and the cap 1539. In the illustrated embodiment, the indicator 1560 includes a first indicator region or portion 1562 and a second indicator region or portion 1564 each having at last one different visual characteristic from one another. For example, in some embodiments the first indicator portion 1562 can be a first color (e.g., red) while the second indicator portion 1564 can be a different second color (e.g., green). In some embodiments, the second indicator portion 1564 is at least partially nested within the first indicator portion 1562 in the vacuum-off position (e.g., as best seen in FIG. 15G). The first indicator portion 1562 (e.g., a lower end portion thereof) can be coupled to the flexible member 1534 while the second indicator portion 1564 (e.g., an upper end portion thereof) can be coupled to the cap 1539.

When the tip 1426 of the syringe 1500 is not under vacuum, the vacuum indicator 1530 is in the vacuum-off position in which the flexible member 1534 is in the first position and biases the first indicator portion 1562 upward toward the cap 1539 such that the second indicator portion 1564 is positioned adjacent the transparent tube 1537—and thus visible to the user—as shown in FIGS. 15C and 15E. At the same time, the first indicator portion 1562 at least partially surrounds the second indicator portion 1564 such that the second indicator portion 1564 is obscured and not visible to the user in the vacuum-off position. When the tip 1426 of the syringe 1500 is under vacuum, the vacuum indicator 1530 is in the vacuum-on position in which the vacuum pulls the flexible member 1534 into/toward the opening 1461, thereby pulling the first indicator portion 1562 downward away from the cap 1539 and the second indicator portion 1564 such that the second indicator portion 1564 is visible through the transparent tube 1537, as shown in FIGS. 15D and 15F. At the same time, the first indicator portion 1562 is obscured by the non-transparent base 1535 in the vacuum-on position. In this manner, the first and second indicator portions 1562, 1564 are configured to provide a visual indication to the user of whether the syringe 1500 (and the coupled assembly 700 of FIG. 7) is under vacuum or not.

In some embodiments, alternatively to or in addition to using the flexible member 1534 to bias the first indicator portion 1562 to the vacuum-off position, the vacuum indicator 1530 can include a biasing member configured to bias the first indicator portion 1562 to the vacuum-off position. FIG. 16, for example, is an enlarged side cross-sectional view of the vacuum indicator 1530 further including a biasing member 1666, such as a spring, operably coupled between the cap 1539 and the first indicator portion 1562 in accordance with embodiments of the present technology. In the illustrated embodiment, the biasing member 1666 has an upper end portion 1667 coupled to the cap 1539 (e.g., a spring-mount thereof) and a lower end portion 1668 coupled to the first indicator portion 1562 (e.g., a spring mount thereof). In some embodiments the biasing member 1666 can extend at least partially through an internal channel 1669 of the second indicator portion 1564. When the tip 1426 of the syringe 1500 is not under vacuum, the biasing member 1666 can bias the first indicator portion 1562 toward the cap 1539 to the vacuum-off position. When the tip 1426 of the syringe 1500 is under vacuum, the flexible member 1534 can pull the first indicator portion 1562 toward the opening 1461 against the biasing force of the biasing member 1666 to the vacuum-on position.

### IV. FURTHER EXAMPLES

The following examples are illustrative of several embodiments of the present technology:

1. An automatically-locking syringe, comprising:
a barrel;
a plunger slidably positioned within the barrel, wherein the plunger is movable between a depressed position and a withdrawn position, and wherein the plunger includes a lock feature; and
a lock plate coupled to the barrel and having an opening extending therethrough, wherein the plunger is slidably positioned in the opening, wherein the lock plate includes a biasing member configured to bias the lock plate to a locking position, and wherein—
   when the plunger is moved from the depressed position to the withdrawn position, the lock feature is configured to engage the lock plate to drive the lock plate to a position away from the locking position to thereby permit the lock feature to pass through the opening, and
   after the lock feature passes through the opening, the biasing member is configured to drive the lock plate to the locking position to inhibit movement of the plunger from the withdrawn position to the depressed position.

2. The syringe of example 1 wherein the lock feature includes a stop surface extending generally perpendicular to a longitudinal axis of the syringe, and wherein the lock plate is configured to engage the stop surface when (a) the lock plate is in the locking position and (b) the plunger is in the withdrawn position.

3. The syringe of example 1 or example 2, further comprising a button coupled to the lock plate, wherein the button is actuatable to move the lock plate away from the locking position to permit movement of the plunger from the withdrawn position to the depressed position.

4. The syringe of any one of examples 1-3 wherein the biasing member includes at least one of a spring and a living hinge.

5. The syringe of any one of examples 1-3 wherein the biasing member is an arm hingedly coupled to the locking plate.

6. The syringe of any one of examples 1-5 wherein the lock feature is one of a plurality of lock features positioned along a longitudinal axis of the plunger.

7. The syringe of example 6 wherein—
when the plunger is moved from the depressed position to the withdrawn position, individual ones of the lock features are configured to sequentially engage the lock plate to drive the lock plate to the position away from the locking position to thereby permit the lock feature to pass through the opening, and
after the individual ones of the lock features sequentially pass through the opening, the biasing member is configured to drive the lock plate to the locking position to inhibit movement of the plunger from the withdrawn position to the depressed position.

8. The syringe of example 6 or example 7, further comprising a locking mechanism configured to engage the lock plate to lock the lock plate in the position away from the locking position.

9. The syringe of any one of examples 1-8 wherein the barrel has a volume of about 60 cc or greater.

10. An automatically-locking syringe, comprising:
a barrel including a flange;
a plunger slidably positioned within the barrel, wherein the plunger is aligned along a longitudinal axis, and wherein the plunger is movable along the longitudinal axis between a depressed position and a withdrawn position;
a lock member coupled to the plunger, wherein the lock member includes a body and a first arm hingedly coupled to the body, and wherein the first arm is configured to be biased at least partially outwardly away from the longitudinal axis of the plunger to a locking position; and
an actuator including a second arm, wherein the actuator is movable between a first position and a second position, and wherein the second arm is configured to engage the first arm in the second position to drive the first arm inwardly toward the longitudinal axis away from the locking position.

11. The syringe of example 10 wherein the first arm is configured to engage the flange of the barrel to inhibit movement of the plunger from the withdrawn position to the depressed position when (a) the actuator is in the first position and (b) the plunger is in the withdrawn position.

12. The syringe of example 10 or example 11 wherein the lock member includes a third arm hingedly coupled to the body, wherein the third arm is configured to be biased at least partially outwardly away from the longitudinal axis of the plunger to a locking position, wherein the actuator includes a fourth arm, and wherein the fourth arm is configured to engage the third arm in the second position to drive the third arm inwardly toward the longitudinal axis away from the locking position.

13. The syringe of example 12 wherein the first arm is configured to be biased at least partially outwardly away from the longitudinal axis in a first direction, and wherein the third arm is configured to be biased at least partially outwardly away from the longitudinal axis in a second direction opposite the first direction.

14. The syringe of example 12 or example 13 wherein the first arm and the third arm have an identical size and shape, and wherein the second arm and the fourth arm have an identical size and shape.

15. The syringe of any one of examples 12-14 wherein the third arm is configured to engage the flange of the barrel to inhibit movement of the plunger from the withdrawn position to the depressed position when (a) the actuator is in the first position and (b) the plunger is in the withdrawn position.

16. A clot treatment system, comprising:
a catheter;
a pressure source configured to generate vacuum pressure; and
a tubing subsystem configured to fluidly connect the catheter to the pressure source, wherein the tubing subsystem includes a vacuum indicator configured to provide an indication that the catheter is under vacuum pressure.

17. The clot treatment system of example 16 wherein the tubing subsystem includes an aperture, wherein the vacuum indicator includes a flexible member positioned over the aperture, and wherein the flexible member is configured to deform about the opening when the catheter is under vacuum pressure to provide the indication that the catheter is under vacuum pressure.

18. The clot treatment system of example 17 wherein the vacuum indicator includes a housing positioned around the flexible member and configured to inhibit excessive deformation of the flexible member when the catheter is under positive pressure.

19. The clot treatment device of any one of examples 16-18, wherein the pressure source is an automatically-locking syringe.

20. The clot treatment device of claim 16 wherein—
the tubing subsystem includes a valve, a first tubing section, a fluid control device, and a second tubing subsystem,
the valve fluidly connects the catheter to the first tubing section,
the first tubing section is fluidly connected between the valve and the fluid control device,
the second tubing system fluidly connects the fluid control device to the pressure source, and
the vacuum indicator is fluidly connected between the valve and the fluid control device.

21. A syringe, comprising:
a barrel having a tip, wherein the tip includes an opening;
a plunger movable through the barrel to generate vacuum pressure within the barrel; and a vacuum indicator positioned over the opening in the tip, wherein the vacuum indicator includes a transparent tube and an indicator movably positioned within the tube, wherein the indicator includes a first region having a first visual characteristic and a second region having a second visual characteristic different than the first visual characteristic, and wherein—
   when the barrel is not under vacuum pressure, the first region is configured to be positioned adjacent the tube; and
   when the barrel is under vacuum pressure, the second region is configured to be positioned adjacent the tube.

22. The syringe of example 21 wherein the vacuum indicator further includes an opaque cap over the tube, wherein the indicator is movably positioned within the cap and the tube, and wherein—
when the barrel is not under vacuum pressure, the second region is configured to be positioned adjacent the cap; and
when the barrel is under vacuum pressure, the first region is configured to be positioned adjacent the cap.

23. The syringe of example 22 wherein the vacuum indicator further includes a biasing member coupled between the indicator and the cap, wherein the biasing member is configured to bias the indicator to position the first region adjacent the tube when the barrel is not under vacuum pressure.

24. The syringe of any one of examples 21-23 wherein the vacuum indicator further includes a flexible member positioned over the opening in the tip and coupled to the indicator.

25. The syringe of example 24 wherein the flexible member is configured to deform about the opening when the catheter is under vacuum pressure.

26. The syringe of example 24 or example 25 wherein the vacuum indicator further includes an opaque cap over the tube, wherein the first portion of the indicator is coupled to the flexible member, and wherein the second portion of the indicator is coupled to the cap.

27. The syringe of any one of examples 21-26 wherein the vacuum indicator further includes a flexible member positioned over the opening in the tip and a biasing member, wherein the first portion of the indicator is coupled to the biasing member, and wherein the second portion of the indicator is coupled to the flexible member.

28. The syringe of any one of examples 21-27 wherein the first and second portions of the indicator are movable relative to one another, and wherein the second indicator is at least partially nested within the second indicator.

### V. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An automatically-locking syringe, comprising:
a barrel;
a plunger slidably positioned within the barrel, wherein the plunger is movable between a depressed position and a withdrawn position, and wherein the plunger includes a lock feature; and
a lock plate coupled to the barrel and having an opening extending therethrough, wherein the plunger is slidably positioned in the opening, wherein the lock plate includes a biasing member configured to bias the lock plate to a locking position, and wherein—
when the plunger is moved from the depressed position to the withdrawn position, the lock feature is configured to engage the lock plate to drive the lock plate to a position away from the locking position to thereby permit the lock feature to pass through the opening, and
after the lock feature passes through the opening, the biasing member is configured to drive the lock plate to the locking position to inhibit movement of the plunger from the withdrawn position to the depressed position.

2. The syringe of claim 1 wherein the lock feature includes a stop surface extending generally perpendicular to a longitudinal axis of the syringe, and wherein the lock plate is configured to engage the stop surface when (a) the lock plate is in the locking position and (b) the plunger is in the withdrawn position.

3. The syringe of claim 1, further comprising a button coupled to the lock plate, wherein the button is actuatable to move the lock plate away from the locking position to permit movement of the plunger from the withdrawn position to the depressed position.

4. The syringe of claim 1 wherein the biasing member includes at least one of a spring and a living hinge.

5. The syringe of claim 1 wherein the biasing member is an arm hingedly coupled to the locking plate.

6. The syringe of claim 1 wherein the lock feature is one of a plurality of lock features positioned along a longitudinal axis of the plunger.

7. The syringe of claim 6 wherein—
when the plunger is moved from the depressed position to the withdrawn position, individual ones of the lock features are configured to sequentially engage the lock plate to drive the lock plate to the position away from the locking position to thereby permit the lock feature to pass through the opening, and
after the individual ones of the lock features sequentially pass through the opening, the biasing member is configured to drive the lock plate to the locking position to inhibit movement of the plunger from the withdrawn position to the depressed position.

8. The syringe of claim 6, further comprising a locking mechanism configured to engage the lock plate to lock the lock plate in the position away from the locking position.

9. The syringe of claim 1 wherein the barrel has a volume of about 60 cc or greater.

10. An automatically-locking syringe, comprising:
a barrel including a flange;
a plunger slidably positioned within the barrel, wherein the plunger is aligned along a longitudinal axis, and wherein the plunger is movable along the longitudinal axis between a depressed position and a withdrawn position;
a lock member coupled to the plunger, wherein the lock member includes a body and a first arm hingedly coupled to the body, and wherein the first arm is configured to be biased at least partially outwardly away from the longitudinal axis of the plunger to a locking position; and
an actuator including a second arm, wherein the actuator is movable between a first position and a second position, and wherein the second arm is configured to engage the first arm in the second position to drive the first arm inwardly toward the longitudinal axis away from the locking position.

11. The syringe of claim 10 wherein the first arm is configured to engage the flange of the barrel to inhibit movement of the plunger from the withdrawn position to the depressed position when (a) the actuator is in the first position and (b) the plunger is in the withdrawn position.

12. The syringe of claim 10 wherein the lock member includes a third arm hingedly coupled to the body, wherein the third arm is configured to be biased at least partially outwardly away from the longitudinal axis of the plunger to a locking position, wherein the actuator includes a fourth arm, and wherein the fourth arm is configured to engage the third arm in the second position to drive the third arm inwardly toward the longitudinal axis away from the locking position.

13. The syringe of claim 12 wherein the first arm is configured to be biased at least partially outwardly away from the longitudinal axis in a first direction, and wherein the third arm is configured to be biased at least partially outwardly away from the longitudinal axis in a second direction opposite the first direction.

14. The syringe of claim 12 wherein the first arm and the third arm have an identical size and shape, and wherein the second arm and the fourth arm have an identical size and shape.

15. The syringe of claim 12 wherein the third arm is configured to engage the flange of the barrel to inhibit movement of the plunger from the withdrawn position to the depressed position when (a) the actuator is in the first position and (b) the plunger is in the withdrawn position.

16. A clot treatment system, comprising:
a catheter;
a pressure source configured to generate vacuum pressure; and
a tubing subsystem configured to fluidly connect the catheter to the pressure source, wherein the tubing subsystem includes a vacuum indicator configured to provide an indication that the catheter is under vacuum pressure.

17. The clot treatment system of claim 16 wherein the tubing subsystem includes an aperture, wherein the vacuum indicator includes a flexible member positioned over the aperture, and wherein the flexible member is configured to deform about the opening when the catheter is under vacuum pressure to provide the indication that the catheter is under vacuum pressure.

18. The clot treatment system of claim 17 wherein the vacuum indicator includes a housing positioned around the flexible member and configured to inhibit excessive deformation of the flexible member when the catheter is under positive pressure.

19. The clot treatment device of claim 16, wherein the pressure source is an automatically-locking syringe.

20. The clot treatment device of claim 16 wherein—
the tubing subsystem includes a valve, a first tubing section, a fluid control device, and a second tubing subsystem,
the valve fluidly connects the catheter to the first tubing section,
the first tubing section is fluidly connected between the valve and the fluid control device,
the second tubing system fluidly connects the fluid control device to the pressure source, and
the vacuum indicator is fluidly connected between the valve and the fluid control device.

21. A syringe, comprising:
a barrel having a tip, wherein the tip includes an opening;
a plunger movable through the barrel to generate vacuum pressure within the barrel; and
a vacuum indicator positioned over the opening in the tip, wherein the vacuum indicator includes a transparent tube and an indicator movably positioned within the tube, wherein the indicator includes a first portion having a first visual characteristic and a second portion having a second visual characteristic different than the first visual characteristic, and wherein—
when the barrel is not under vacuum pressure, the first portion is configured to be positioned adjacent the tube; and
when the barrel is under vacuum pressure, the second portion is configured to be positioned adjacent the tube.

22. The syringe of claim 21 wherein the vacuum indicator further includes an opaque cap over the tube, wherein the indicator is movably positioned within the cap and the tube, and wherein—
when the barrel is not under vacuum pressure, the second portion is configured to be positioned adjacent the cap; and
when the barrel is under vacuum pressure, the first portion is configured to be positioned adjacent the cap.

23. The syringe of claim 22 wherein the vacuum indicator further includes a biasing member coupled between the indicator and the cap, wherein the biasing member is configured to bias the indicator to position the first portion adjacent the tube when the barrel is not under vacuum pressure.

24. The syringe of claim 21 wherein the vacuum indicator further includes a flexible member positioned over the opening in the tip and coupled to the indicator.

25. The syringe of claim 24 wherein the flexible member is configured to deform about the opening when the catheter is under vacuum pressure.

26. The syringe of claim 24 wherein the vacuum indicator further includes an opaque cap over the tube, wherein the first portion of the indicator is coupled to the flexible member, and wherein the second portion of the indicator is coupled to the cap.

27. The syringe of claim 21 wherein the vacuum indicator further includes a flexible member positioned over the opening in the tip and a biasing member, wherein the first portion of the indicator is coupled to the biasing member, and wherein the second portion of the indicator is coupled to the flexible member.

28. The syringe of claim 21 wherein the first and second portions of the indicator are movable relative to one another, and wherein the second indicator is at least partially nested within the second indicator.
